(19) United States
(12) Patent Application Publication
(10) Pub. No.: US 2013/0345289 A1
(43) Pub. Date: Dec. 26, 2013

(54) ADULT STEM CELLS, MOLECULAR SIGNATURES, AND APPLICATIONS IN THE EVALUATION, DIAGNOSIS, AND THERAPY OF MAMMALIAN CONDITIONS

(71) Applicants: Frederick Oliver Cope, Westerville, OH (US); Michael Sherman Blue, Powell, OH (US)

(72) Inventors: Frederick Oliver Cope, Westerville, OH (US); Michael Sherman Blue, Powell, OH (US)

(21) Appl. No.: 13/939,491

(22) Filed: Jul. 11, 2013

Related U.S. Application Data
(63) Continuation of application No. 13/458,070, filed on Apr. 27, 2012, now abandoned.
Application No. 13/458,070 is a division of application No. 12/217,426, filed on Jul. 3, 2008, now abandoned.

(51) Int. Cl. C12Q 1/68

(52) U.S. Cl. CPC C12Q 1/6888
USPC 514/44 A; 506/9

(57) ABSTRACT

The present invention relates to the identification of a stem cell-specific signature or signatures composed of protein and/or nucleic acid markers expressed by virtue of the position of a cell or cells in the time line of its/their development and the impact of the cells' environment on this signature as it relates to the cells' stem cell potential. The composition and combination of these signatures provides a means of identifying, manipulating and differentiating said adult stem cells and thus, their acquisition and utilization in research, diagnosis, and therapy of normal and pathological conditions.

### RELATED APPLICATION DATA

This application is a continuation of U.S. application Ser. No. 13/458,070, filed Apr. 27, 2012, which is a divisional of U.S. application Ser. No. 12/217,426, filed Jul. 3, 2008, which are hereby incorporated in their entirety herein by reference.

### TECHNICAL FIELD OF THE INVENTION

The present invention is in the field of cell biology, and more specifically, stem cell biology wherein the invention relates to the identification of stem cells, and their stem cell-specific signature or signatures composed of protein and/or nucleic acid markers expressed by virtue of the position of a cell or cells relative to the potential of its/their own fate, to with the composition and combination of these markers provide a means of identifying said adult stem cells and thus, their acquisition and utilization in research, evaluation, diagnosis, and therapy of normal and pathological conditions.

### BACKGROUND OF THE INVENTION

All publications, patent applications, patents, interne web pages and other references mentioned herein are expressly incorporated by reference in their entirety. When the definitions of terms in incorporated references appear to differ from the definitions provided in the present teachings, the definitions provided in the present teachings shall control.

Stem cells, isolated from tissues derived, starting from the perinatal period and thereafter, are undifferentiated or non-terminally differentiated cells that retain a biological signature that is prospectively integrated into the maturation process so as to provide a reservoir of cells capable of self renewal and which, through micro-environmental influences, can either retain such pluri-potency or stochastically differentiate into tissues that aid in the retention of a healthy tissue or organ architecture and/or a healthy functioning individual. The ability of cells to retain this pluri-potency has been ascribed to numerous factors, yet, without the specificity of a broad signature that defines the capacity to maintain and subsequently differentiate these cells “at will”, into clinically or commercially viable commodities that may be applied to research, specific commercial endeavors, e.g. developing drugs, rendering the cells themselves into therapeutically applicable tissues, or even more specifically, determining the role or roles of a signature molecule or molecules derived from these cells that may be used substitutively, therapeutically, in vivo, or as differentiating agents in vitro (Slavin S, Kurkalli B G, Karussis D. The potential use of adult stem cells for the treatment of multiple sclerosis and other neurodegenerative disorders. Clin Neurol Neurosurg. 2008 Mar. 5; Epub PMID: 18325660; Sahin M B, Schwartz R E, Buckley S M, Heremans Y, Chase L, Hu W S, Verfaillie C M. Isolation and characterization of a novel population of progenitor cells from unmanipulated rat liver. Liver Transpl. 2008 March; 14(3):333-45; King C C, Beattie G M, Lopez A D, Hayek A. Generation of definitive endoderm from human embryonic stem cells cultured in feeder layer-free conditions. Regen Med. 2008 March; 3(2):175-80; Fransioli J, Bailey B, Gude N A, Cottage C T, Muraski J A, Emmanuel G, Wu W, Alvarez R, Rubio M, Ottolenghi S, Schaefer E, Sussman M A. Evolution of The c-kit Positive Cell Response to Pathological Challenge in the Myocardium. Stem Cells. 2008 Feb. 28; Epub PMID: 18308948]; Park Y B, Kim Y Y, Oh S K, Chung S G, Ku S Y, Kim S H, Choi Y M, Moon S Y. Alterations of proliferative and differentiation potentials of human embryonic stem cells during long-term culture. Exp Mol Med. 2008 Feb. 29; 40(1):98-108; Agarwal S, Holton K L, Lanza R. Efficient Differentiation of Functional Hepatocytes from Human Embryonic Stem Cells. Stem Cells. 2008 Feb. 21; Epub PMID; Garber K. Epithelial-to-mesenchymal transition is important to metastasis, but questions remain. J Natl Cancer Inst. 2008 Feb. 20; 100(4):232-3, 239; Toyooka Y, Shimosato D, Murakami K, Takahashi K, Niwa H. Identification and characterization of subpopulations in undifferentiated ES cell culture. Development. 2008 March; 135(5):909-18; Kerr C L, Hill C M, Blumenthal P D, Gearhart J D. Expression of pluripotent stem cell markers in the human fetal ovary. Hum Reprod. 2008 March; 23(3):589-99; Tsuneyoshi N, Sumi T, Onda H, Nojima H, Nakatsuji N, Suemori H. PRDM14 suppresses expression of differentiation marker genes in human embryonic stem cells. Biochem Biophys Res Commun. 2008 Mar. 21; 367(4):899-905; Kolodziejska K M, Ashraf H N, Nagy A, Bacon A, Frampton J, Xin H B, Kotlikoff M I, Husain M. c-Myb Dependent Smooth Muscle Cell Differentiation. Circ Res. 2008 Jan. 10; Epub PMID: 18187733; Dhara S K, Hasneen K, Machacek D W, Boyd N L, Rao R R, Slice S L. Human neural progenitor cells derived from embryonic stem cells in feeder-free cultures. Differentiation. 2008 Jan. 3; Epub PMID: 18177420; Cholette J M, Blumberg N, Phipps R P, McDermott M P, Gettings K F, Lerner N B. Developmental changes in soluble CD40 ligand. J Pediatr. 2008 January; 152(1):50-4, 54.e1; Nadri S, Soleimani M, Kiani J, Atashi A, Izadpanah R. Multipotent mesenchymal stem cells from adult human eye conjunctiva stromal cells. Differentiation. 2008 March; 76(3):223-31; Barker N, Clevers H. Tracking down the stem cells of the intestine: strategies to identify adult stem cells. Gastroenterology. 2007 December; 133(6): 1755-60; Lakshmipathy U, Hart R P. Concise review: MicroRNA expression in multipotent mesenchymal stromal cells. Stem Cells. 2008 February; 26(2): 356-63; Kerr C L, Hill C M, Blumenthal P D, Gearhart J D. Expression of pluripotent stem cell markers in the human fetal testis. Stem Cells. 2008 February; 26(2):412-21; Babaie Y, Herwig R, Greber B, Brink T C, Wruck W, Groth D, Lehrach H, Burdon T, Adjaye J. Analysis of Oct4-dependent transcriptional networks regulating self-renewal and pluripotency in human embryonic stem cells. Stem Cells. 2007 February; 25(2):500-10; Ai C, Todorov I, Slovak M L, Digiusto D, Forman S J, Shih C C. Human marrow-derived mesodermal progenitor cells generate insulin-secreting islet-like clusters in vivo. Stem Cells Dev. 2007 October; 16(5):757-70; Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A. Induced pluripotent stem cell lines derived from human somatic cells. Science. 2007 Dec. 21; 318(5858):1917-20; Abzhanov A, Rodda S J, McMahon A P, Tabin C J. Regulation of skeletogenic differentiation in cranial dermal bone. Development. 2007 September; 134(17):3133-44; Mokrý; J, Karbanová J, Cizková D, Pazour J, FAD S, Osterreicher J. Differentiation of neural stem cells into cells of oligodendroglial lineage. Acta Medica (Hradec Kralove). 2007; 50(1):35-41; Peiffer I, Belhomme D, Barbet R, Haydont V, Zhou Y P, Fortunel N O, Li M, Hatzfeld A, Fabiani J N, Hatzfeld J A. Simultaneous differentiation of endothelial and trophoblastic cells derived from human embryonic stem cells. Stem Cells Dev. 2007 June; 16(3):393-402; Stec M, Weglarczyk K, Baran J, Zuba E, Mytar B, Pryjma J, Zembala M. Expansion and differentiation of CD14(+)+CD16(−) and CD14(+)+CD16(+) human monocyte subsets from cord blood CD34(+) hematopoietic progenitors. J Leukoc Biol. 2007 September; 82(3):594-602; Lei Z, Yongda L, Jun M, Yingyu S, Shaoju Z, Xinwen Z, Mingxue Z. Culture and neural differentiation of rat bone marrow mesenchymal stem cells in vitro. Cell Biol Int. 2007 September; 31(9):916-23; Wang Z X, Teh C H, Kueh J L, Lufkin T, Robson P, Stanton L W. Oct4 and Sox2 directly regulate expression of another pluripotency transcription factor, Zfp206, in embryonic stem cells. J Biol Chem. 2007 Apr. 27; 282(17):12822-30; Lengner C J, Camargo F D, Hochedlinger K, Welstead G G, Zaidi S, Gokhale S, Scholer H R, Tomilin A, Jaenisch R. Oct4 expression is not required for mouse somatic stem cell self-renewal. Cell Stem Cell. 2007 Oct. 11; 1(4):403-415; Donnenberg V S, Luketich J D, Landreneau R J, DeLoia J A, Basse P, Donnenberg A D. Tumorigenic epithelial stein cells and their normal counterparts. Ernst Schering Found Symp Proc. 2006; 5:245-63; Western P, Maldonado-Saldivia J, van den Bergen J, Hajkova P, Saitou M, Barton S, Surani M A. Analysis of Esg1 expression in pluripotent cells and the germ line reveals similarities with Oct4 and Sox2 and differences between human pluripotent cell lines. Stem Cells. 2005 November-December; 23 (10): 1436-42).

Ultimately, it will be necessary to differentiate and expand stem cells into specific cell lineages and in sufficient quantities so as to be commercially and clinically acceptable. From the aspect of cell-based therapies, cells suitable for this therapeutic approach need to 1) provide robust and persistent engraftment to repair injury or correct genetic disease; 2) undergo tissue specific differentiation, either prior to transplantation or in vivo, and; 3) be expandable to the scale required for clinical application. Prior research on individual growth factors, signaling molecules, or extracellular matrix components has been insufficient to define the factors and conditions required for the production of differentiated cells in sufficient number for clinical use or to stimulate appropriate differentiation in situ. Advances in stem cell biology, including the identification of key molecules regulating self-renewal and differentiation and the establishment of new model systems, provide opportunities to address this roadblock and to stimulate new approaches to providing clinically and economically valuable materials.

For example, critical elements that control the proliferation versus differentiation choices of resident heart, vascular, lung, and blood stem or progenitor cells need to be understood. Such information is of paramount importance to devising and successfully implementing cell-based therapies for heart, vascular, lung, and blood diseases, cancer, or alternatively, therapies based on the linage signatures where these signatures are discerned to be specific molecules that can be implemented with predictable cellular outcome. Cell-based therapies or imposition of specific molecular signatures of these cells could impact treatment of diseases such as myocardial infarction, heart failure, end-stage emphysema, and the repair of atherosclerotic vessels.

The expression of numerous markers, including transcription factors, integrating genetic factors, and other protein markers have been sufficient to carry the stem cell field forward to a limited degree. These markers include Oct-3/4, SOX2, NANOG, SSEA3, SSEA4, SSEA1, MART, CD34, and others. In particular, Oct-3/4 has been shown to be particularly important in defining the “stemness” of all stem cells, including the pluri-potency of the adult stem cell lineages. However, recent evidence indicates that even this marker is insufficient to monolithically support the concept of pluri-potency (Kerr C L, Hill C M, Blumenthal P D, Gearhart J D. Expression of pluripotent stem cell markers in the human fetal ovary. Hum Reprod. 2008 March; 23(3):589-99; Nadri S, Soleimani M, Kiani J, Atashi A, Izadpanah R. Multipotent mesenchymal stem cells from adult human eye conjunctiva stromal cells. Differentiation. 2008 March; 76(3):223-31; Lakshmipathy U, Hart R P. Concise review: MicroRNA expression in multipotent mesenchymal stromal cells. Stem Cells. 2008 February; 26(2):356-63; Kerr C L, Hill C M, Blumenthal P D, Gearhart J D. Expression of pluripotent stem cell markers in the human fetal testis. Stem Cells. 2008 February; 26(2):412-21; Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A. Induced pluripotent stem cell lines derived from human somatic cells. Science. 2007 Dec. 21; 318(5858):1917-20; Lei Z, Yongda L, Jun M, Yingyu S, Shaoju Z, Xinwen Z, Mingxue Z. Culture and neural differentiation of rat bone marrow mesenchymal stem cells in vitro. Cell Biol Int. 2007 September; 31(9):916-23; Wang Z X Teh C H, Kueh J L, Lufkin T, Robson P, Stanton L W. Oct4 and Sox2 directly regulate expression of another pluripotency transcription factor, Zfp206, in embryonic stem cells. J Biol Chem. 2007 Apr. 27; 282(17):12822-30; Lengner C J, Camargo F D, Hochedlinger K, Welstead G G, Zaidi S, Gokhale S, Scholer H R, Tomilin A, Jaenisch R. Oct-4 expression is not required for mouse somatic stem cell self-renewal. Cell Stem Cell. 2007 Oct. 11; 1(4):403-415; Donnenberg V S, Luketich J D, Landreneau R J, DeLoia J A, Basse P, Donnenberg A D. Tumorigenic epithelial stem cells and their normal counterparts. Ernst Schering Found Symp Proc. 2006; 5:245-63; Western P, Maldonado-Saldivia J, van den Bergen J, Hajkova P, Saitou M, Barton S, Surani M A. Analysis of Esg1 expression in pluripotent cells and the germ line reveals similarities with Oct4 and Sox2 and differences between human pluripotent cell lines. Stem Cells. 2005 November-December; 23(10):1436-42).

Thus, a focus on elucidating alternative factors that define and direct the differentiation of stem or progenitor cells, especially of adult origin, into defined pathways or cell lineages and maintaining that differentiated state is the central matter to providing copious amounts of cells that can reliably differentiate into clinically utilizable materials or alternatively, to providing heretofore unknown compositions of molecules that reliably define valuable endpoints or induce regeneration that may be applied to expanding normal tissues or positively altering pathological states to promote healing or desirable tissue or cellular changes ((Slavin S, Kurkalli B G, Karussis D. The potential use of adult stem cells for the treatment of multiple sclerosis and other neurodegenerative disorders. Clin Neurol Neurosurg. 2008 Mar. 5; Epub PMID: 18325660; Sahin M B, Schwartz R E, Buckley S M, Heremans Y, Chase L, Hu W S, Verfaillie C M. Isolation and characterization of a novel population of progenitor cells from unmanipulated rat liver. Liver Transpl. 2008 March; 14(3):333-45; King C C, Beattie G M, Lopez A D, Hayek A. Generation of definitive endoderm from human embryonic stem cells cultured in feeder layer-free conditions. Regen Med. 2008 March; 3(2):175-80; Fransioli J, Bailey B, Gude N A, Cottage C T, Muraski J A, Emmanuel G, Wu W, Alvarez R, Rubio M, Ottolenghi S, Schaefer E, Sussman M A. Evolution of The c-kit Positive Cell Response to Pathological Challenge in the Myocardium. Stem Cells. 2008 Feb. 28; Epub PMID: 18308948; Park Y B, Kim Y Y, Oh S K, Chung S G, Ku S Y, Kim S H, Choi Y M, Moon S Y. Alterations of proliferative and differentiation potentials of human embryonic stem cells during long-term culture. Exp Mol Med. 2008 Feb. 29; 40(1):98-108; Agarwal S, Holton K L, Lanza R. Efficient Differentiation of Functional Hepatocytes from Human Embryonic Stem Cells. Stem Cells. 2008 Feb. 21; Epub PMID; Garber K. Epithelial-to-mesenchymal transition is important to metastasis, but questions remain. J Natl Cancer Inst. 2008 Feb. 20; 100(4):232-3, 239; Toyooka Y, Shimosato D, Murakami K, Takahashi K, Niwa H. Identification and characterization of subpopulations in undifferentiated ES cell culture. Development. 2008 March; 135(5):909-18; Kerr C L, Hill C M, Blumenthal P D, Gearhart J D. Expression of pluripotent stem cell markers in the human fetal ovary. Hum Reprod. 2008 March; 23(3):589-99; Tsuneyoshi N, Sumi T, Onda H, Nojima H, Nakatsuji N, Suemori H. PRDM14 suppresses expression of differentiation marker genes in human embryonic stem cells. Biochem Biophys Res Commun. 2008 Mar. 21; 367(4):899-905; Kolodziejska K M, Ashraf H N, Nagy A, Bacon A, Frampton J, Xin H B, Kotlikoff M I, Husain M. c-Myb Dependent Smooth Muscle Cell Differentiation. Circ Res. 2008 Jan. 10; Epub PMID: 18187733; Dhara S K, Hasneen K, Machacek D W, Boyd N L, Rao R R, Stice S L. Human neural progenitor cells derived from embryonic stem cells in feeder-free cultures. Differentiation. 2008 Jan. 3; Epub PMID: 18177420; Cholette J M, Blumberg N Phipps R P, McDermott M P, Gettings K F, Lerner N B. Developmental changes in soluble CD40 ligand. J Pediatr. 2008 January; 152(1):50-4, 54.e1; Nadri S, Soleimani M, Kiani J, Atashi A, Izadpanah R. Multipotent mesenchymal stem cells from adult human eye conjunctiva stromal cells. Differentiation. 2008 March; 76(3):223-31; Barker N, Clevers H. Tracking down the stem cells of the intestine: strategies to identify adult stem cells. Gastroenterology. 2007 December; 133(6):1755-60; Lakshmipathy U, Hart R P. Concise review: MicroRNA expression in multipotent mesenchymal stromal cells. Stem Cells. 2008 February; 26(2):356-63; Kerr C L, Hill C M, Blumenthal P D, Gearhart J D. Expression of pluripotent stem cell markers in the human fetal testis. Stem Cells. 2008 February; 26(2):412-21; Babaie Y, Herwig R, Greber B, Brink T C, Wruck W, Groth D, Lehrach H, Burdon T, Adjaye J. Analysis of Oct4-dependent transcriptional networks regulating self-renewal and pluripotency in human embryonic stem cells. Stem Cells. 2007 February; 25(2):500-10; Ai C, Todorov I, Slovak M L, Digiusto D, Forman S J, Shih C C. Human marrow-derived mesodermal progenitor cells generate insulin-secreting islet-like clusters in vivo. Stem Cells Dev. 2007 October; 16(5): 757-70; Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A. Induced pluripotent stem cell lines derived from human somatic cells. Science. 2007 Dec. 21; 318(5858):1917-20; Abzhanov A, Rodda S J, McMahon A P, Tabin C J. Regulation of skeletogenic differentiation in cranial dermal bone. Development. 2007 September; 134(17):3133-44; Mokrý J, Karbanová J, Cizková D, Pazour J, Filip S, Osterreicher J. Differentiation of neural stem cells into cells of oligodendroglial lineage. Acta Medica (Hradec Kralove). 2007; 50(1):35-41; Peiffer I, Belhomme D, Barbet R, Haydont V, Zhou Y P, Fortunel N O, Li M, Hatzfeld A, Fabiani J N, Hatzfeld J A. Simultaneous differentiation of endothelial and trophoblastic cells derived from human embryonic stem cells. Stem Cells Dev. 2007 June; 16(3):393-402; Stec M, Weglarczyk K, Baran J, Zuba E, Mytar B, Pryjma J, Zembala M. Expansion and differentiation of CD14+CD16(−) and CD14++CD16+ human monocyte subsets from cord blood CD34+ hematopoietic progenitors. J Leukoc Biol. 2007 September; 82(3): 594-602).

Implicit in the description of stem cells is the acknowledgement that stem cells other than embryonic stem cells may be source material for the reliable and safe production of stable end phenotypes. Lei et al, Wang et al, Lengner et al, Donnenberg, et al, and Western et al, and others (Lei Z, Yongda L, Jun M, Yingyu S, Shaoju Z, Xinwen Z, Mingxue Z. Culture and neural differentiation of rat bone marrow mesenchymal stem cells in vitro. Cell Biol Int. 2007 September; 31(9):916-23; Wang Z X, Teh C H, Kueh J L, Lufkin T, Robson P, Stanton L W. Oct4 and Sox2 directly regulate expression of another pluripotency transcription factor, Zfp206, in embryonic stem cells. J Biol Chem. 2007 Apr. 27; 282(17):12822-30; Lengner C J, Camargo F D, Hochedlinger K, Welstead G G, Zaidi S, Gokhale S, Scholer H R, Tomilin A, Jaenisch R. Oct4 expression is not required for mouse somatic stem cell self-renewal. Cell Stem Cell. 2007 Oct. 11; 1(4):403-415; Donnenberg V S, Luketich J D, Landreneau R J, DeLoia J A, Basse P, Donnenberg A D. Tumorigenic epithelial stem cells and their normal counterparts. Ernst Schering Found Symp Proc. 2006; 5:245-63; Western P, Maldonado-Saldivia J, van den Bergen J, Hajkova P, Saitou M, Barton S, Surani M A. Analysis of Esg1 expression in pluripotent cells and the germ line reveals similarities with Oct4 and Sox2 and differences between human pluripotent cell lines. Stem Cells. 2005 November-December; 23(10):1436-42, respectively) teach the use of numerous types of tissue as sources of adult stem cells. In fact, numerous references are made to mesenchymal stem cells. There are, however, several critical observations that have not been made with reference to these sources of adult stem cells. None of the investigators teaches that any of these sources of stem cells produce, immediately at acquisition, a homogeneous population of cells with greater than 6×10⁶ cells available all with pleuripotential capability. As one example of the prior art of adult stem cell isolation, references that cite the wisdom tooth or the deciduous teeth as a source of adult stem cells, specifically require isolation from a preformed tooth ligament or isolation from the dental pulp or isolation of cells or a single cell from the oral ectoderm (Zhang C, Chang J, Sonoyama W, Shi S, Wang C Y. Inhibition of human dental pulp stem cell differentiation by notch signaling. J Dent Res. 2008 March; 87(3):250-5; Suchánek J, Soukup T, Ivancaková R, Karbanová J, Hubková V, Pytlík R, Kucerová L. Human dental pulp stem cells—isolation and long term cultivation. Acta Medica (Hradec Kralove). 2007; 50(3):195-201; Liu Y, Zheng Y, Ding G, Fang D, Zhang C, Bartold P M, Gronthos S, Shi S, Wang S. Periodontal Ligament Stem Cell-mediated Treatment for Periodontitis in Miniature Swine. Stem Cells. 2008 Jan. 31; [Epub PMID: 18238856]; Scheller E L, Chang J, Wang C Y. Wnt/beta-catenin inhibits dental pulp stem cell differentiation. J Dent Res. 2008 February; 87(2):126-30; Morsczeck C, Schmalz G, Reichert T E, Völlner F, Galler K, Driemel O. Somatic stem cells for regenerative dentistry. Clin Oral Investig. 2008 Jan. 3; Epub PMID: 18172700; Ikeda E, Yagi K, Kojima M, Yagyuu T, Ohshima A, Sobajima S, Tadokoro M, Katsube Y, Isoda K, Kondoh M, Kawase M, Go M J, Adachi H, Yokota Y, Kirita T, Ohgushi H. Multipotent cells from the human third molar: feasibility of cell-based therapy for liver disease. Differentiation. 2007 Dec. 17; Epub PMID: 18093227; Yen A H, Sharpe P T Stem cells and tooth tissue engineering. Cell Tissue Res. 2008 January; 331(1):359-372; Wei X Ling J, Wu L, Liu L, Xiao Y. Expression of mineralization markers in dental pulp cells. J Endod. 2007 June; 33(6):703-8; Ballini A, De Frenza G, Cantore S, Papa F, Grano M, Mastrangelo F, Tetè S, Grassi F R. In vitro stem cell cultures from human dental pulp and periodontal ligament: new prospects in dentistry. Int J Immunopathol Pharmacol. 2007 January-March; 20(1):9-16; Ohazama A, Modino S A, Miletich I, Sharpe P T. Stem-cell-based tissue engineering of murine teeth. J Dent Res. 2004 July; 83(7):518-22; Miura M, Gronthos S, Zhao M, Lu B, Fisher L W, Robey P G, Shi S. SHED: stem cells from human exfoliated deciduous teeth. Proc Natl Acad Sci USA. 2003 May 13; 100(10):5807-12; USPTO Applications 20080038770, Cancer stem cells and uses thereof USPTO 20080038238, Human stem cell materials and methods; USPTO 20080033548, Bone tissue engineering by ex vivo stem cells on growth into 3d trabecular metal; USPTO 20080031820, Swine multipotent adult progenitor cells; USPTO 20080025857, Stem cell suitable for transplantation, their preparation and pharmaceutical compositions comprising them; and U.S. Pat. No. 7,332,336, Methods for inducing differentiation of pluripotent cells). Additionally and importantly, none of the prior art defines specific signatures of adult stem cells derived from any anatomical structure, including the oral cavity. Specifially, none of the prior art documents the unique signatures of microRNAs in stem cells as contrasted with any normal differentiated anatomically correct counterpart to validate that such a signature defines the stemness of these cells. MicroRNA or miRs are a group of small non-coding RNA (ncRNA) molecules, distinct from but related to small interfering RNAs (siRNAs), that have been identified in a variety of organisms (Moss E G. MicroRNAs: hidden in the genome. Curr Biol. 2002 Feb. 19; 12(4):R138-40; Smallridge R. A small fortune. Nat Rev Mol Cell Biol. 2001 December; 2(12):867; McManus M T, Sharp P A. Gene silencing in mammals by small interfering RNAs. Nat Rev Genet. 2002 October; 3(10):737-47.). These 19-23 nucleotides (nt), mature single stranded RNAs are transcribed as parts of longer molecules of several kilobases (kb) in length that are processed in the nucleus into hairpin RNAs of 70-100 nt by the double-stranded RNA-specific ribonuclease, “Drosha”. The hairpin RNAs are transported to the cytoplasm, via an exportin-5 dependent mechanism, where they are digested by a second, double-stranded specific ribonuclease called “Dicer”. In animals, single-stranded microRNA binds specific messenger RNA (mRNA) through sequences that are significantly, though not completely, complementary to the target mRNA, mainly to the 3′ untranslated region (3′ UTR). By a mechanism that is not fully characterized, the bound mRNA remains untranslated, resulting in reduced levels of the corresponding protein; alternatively, the bound mRNA can be degraded, resulting in reduced levels of the corresponding transcript. The central dogma of classical biology is that genetic information flows from DNA to RNA to proteins. Therefore “genes” are synonymous with proteins and a gene is defined as a protein-coding region with associated regulatory signals. However, miRs are non-coding RNAs. That is, they do not code for protein products and are the means unto their own regulatory end of cell processes and the regulation of the expression of other genes that may code for mRNA and, thus, proteins. In short, miRs represent an excellent point of exploitation in commercial stem cell production, especially when the linkage for these small molecules to stemness and to the regulatory factors that define stemness remain totally undefined in the prior art (Garofalo M, Quintavalle C, Di Leva G, Zanca C, Romano G, Taccioli C, Liu C G, Croce C M, Condorelli G. MicroRNA signatures of TRAIL resistance in human non-small cell lung cancer. Oncogene. 2008 Feb. 4; Epub PMID: 18246122; Garzon R, Volinia S, Liu C G, Fernandez-Cymering C, Palumbo T, Pichiorri F, Fabbri M, Coombes K, Alder H, Nakamura T, Flomenberg N, Marcucci G, Calin G A, Kornblau S M, Kantarjian H, Bloomfield C D, Andreeff M, Croce C M. MicroRNA signatures associated with cytogenetics and prognosis in acute myeloid leukemia. Blood. 2008 Jan. 10; Epub PMID: 18187662; Yu S L, Chen H Y, Chang G C, Chen C Y, Chen H W, Singh S, Cheng C L, Yu C J, Lee Y C, Chen H S, Su T J. Chiang C C, Li H N, Hong Q S, Su H Y, Chen C C, Chen W J, Liu C C, Chan W K, Chen W J, Li K C, Chen J J, Yang P C. MicroRNA signature predicts survival and relapse in lung cancer. Cancer Cell. 2008 January; 13(1):48-57; Pekarsky Y, Santanam U, Cimmino A, Palamarchuk A, Efanov A, Maximov V, Volinia S, Alder H, Liu C G, Rassenti L, Calin G A, Hagan J P, Kipps T, Croce C M. Tcl1 expression in chronic lymphocytic leukemia is regulated by miR-29 and miR-181. Cancer Res. 2006 Dec. 15; 66(24):11590-3; Calin G A, Croce C M. MicroRNA signatures in human cancers. Nat Rev Cancer. 2006 November; 6(11):857-66; Liu C G, Calin G A, Meloon B, Gamliel N, Sevignani C, Ferracin M, Dumitru C D, Shimizu M, Zupo S, Dono M, Alder H, Bullrich F, Negrini M, Croce C M. An oligonucleotide microchip for genome-wide microRNA profiling in human and mouse tissues. Proc Natl Acad Sci USA. 2004 Jun. 29; 101(26):9740-4; Sood P, Krek A, Zavolan M, Macino G, Rajewsky N. Cell-type-specific signatures of microRNAs on target mRNA expression. Proc Natl Acad Sci USA. 2006 Feb. 21; 103(8):2746-51).

It is not enough to simply imply by assay that a particular marker or miR appears to be important, even if this marker were shown to drive a so-called stem cell to some alternative phenotype. Such observations are stochastic and cannot be said to be a signature and especially one that is predictably useful in identifying, manipulating, or producing clinically valuable tissues or where the marker molecules themselves, may be used diagnosticaly, evaluatively, or therpapeutically. Some current literature does attempt to elucidate potentially unique microRNAs in embryonic stem cells. But no contrasting significance is made by virtue of deriving terminally differentiated tissues via the utilization of microRNA signatures specific to the stem cells or the terminal phenotype or their contrasting miR or marker signatures. Neither is there any attempt to contrast the expression of any so called marker with an anatomical counterpart or to impose on a contrast, the temporal expression of the markers or miRs. So then, how can one claim any expression of a miR as a “signature” component for any pluirpotent cell without such controls or contrasts? (Sinkkonen L, Hugenschmidt T, Berninger P, Gaidatzis D, Mohn F, Artus-Revel C G, Zavolan M, Svoboda P, Filipowicz W. MicroRNAs control de novo DNA methylation through regulation of transcriptional repressors in mouse embryonic stem cells. Nat Struct Mol Biol. 2008 March; 15(3):259-67; Yi R, Poy M N, Stoffel M, Fuchs E. A skin microRNA promotes differentiation by repressing ‘stemness’. Nature. 2008 Mar. 2; Epub PMID: 18311128; Lakshmipathy U, Hart R P. Concise review: MicroRNA expression in multipotent mesenchymal stromal cells. Stem Cells. 2008 February; 26(2):356-63; O'Connell R M, Rao D S, Chaudhuri A A, Boldin M P, Taganov K D, Nicoll J, Paquette R L, Baltimore D. Sustained expression of microRNA-155 in hematopoietic stem cells causes a myeloproliferative disorder. J Exp Med. 2008 Feb. 25; [Epub PMID: 18299402]; Stadler B M, Ruohola-Baker H. Small RNAs: keeping stem cells in line. Cell. 2008 Feb. 22; 132(4):563-6; Morin R D, O'Connor M D, Griffith M, Kuchenbauer F, Delaney A, Prabhu A L, Zhao Y, McDonald H, Zeng T, Hirst M, Eaves C J, Marra M A. Application of massively parallel sequencing to microRNA profiling and discovery in human embryonic stem cells. Genome Res. 2008 Feb. 19; Epub PMID: 18189265; Mizuno Y, Yagi K, Tokuzawa Y, Kanesaki-Yatsuka Y, Suda T, Katagiri T, Fukuda T, Maruyama M, Okuda A, Amemiya T, Kondoh Y, Tashiro H, Okazaki Y. miR-125b inhibits osteoblastic differentiation by down-regulation of cell proliferation. Biochem Biophys Res Commun. 2008 Apr. 4; 368(2):267-72; Yin J Q, Zhao R C, Morris K V. Profiling microRNA expression with microarrays. Trends Biotechnol. 2008 February; 26(2):70-6; Liao R, Sun J, Zhang L, Lou G, Chen M, Zhou D, Chen Z, Zhang S. MicroRNAs play a role in the development of human hematopoietic stem cells. J Cell Biochem. 2008 Jan. 11; Epub PMID: 18189265; Yu F, Yao H, Zhu P, Zhang X Pan Q, Gong C, Huang Y, Hu X, Su F, Lieberman J, Song E. let-7 regulates self renewal and tumorigenicity of breast cancer cells. Cell. 2007 Dec. 14; 131(6):1109-23; Foshay K M, Gallicano G I. Small RNAs, big potential: the role of MicroRNAs in stem cell function. Curr Stem Cell Res Ther. 2007 December; 2(4):264-71; Lakshmipathy U, Love B, Goff L A, Jörnsten R, Graichen R, Hart R P, Chesnut J D. MicroRNA expression pattern of undifferentiated and differentiated human embryonic stem cells. Stem Cells Dev. 2007 December; 16(6):1003-16; Hatfield S, Ruohola-Baker H. microRNA and stem cell function. Cell Tissue Res. 2008 January; 331(1):57-66; Wang Y, Medvid R, Melton C, Jaenisch R, Blelloch R. DGCR8 is essential for microRNA biogenesis and silencing of embryonic stem cell self-renewal. Nat Genet. 2007 March; 39(3):380-5; Georgantas R W 3rd, Hildreth R, Morisot S, Alder J, Liu C G, Heimfeld S, Calin G A, Croce C M, Civin C I. CD34+ hematopoietic stem-progenitor cell microRNA expression and function: a circuit diagram of differentiation control. Proc Natl Acad Sci USA. 2007 Feb. 20; 104(8):2750-5; Anderson C, Catoe H, Werner R. MIR-206 regulates connexin43 expression during skeletal muscle development. Nucleic Acids Res. 2006; 34(20):5863-71; Tang F, Hajkova P, Barton S C, Lao K, Surani M A. MicroRNA expression profiling of single whole embryonic stem cells. Nucleic Acids Res. 2006 Jan. 24; 34(2):e9; Raftopoulou M. microRNA signals cell fate. Nat Cell Biol. 2006 February; 8(2):112; Song L, Tuan R S. MicroRNAs and cell differentiation in mammalian development. Birth Defects Res C Embryo Today. 2006 June; 78(2):140-9; Suh M R, Lee Y, Kim J Y, Kim S K, Moon S H, Lee J Y, Cha K Y, Chung H M, Yoon H S, Moon S Y, Kim V N, Kim K S. Human embryonic stem cells express a unique set of microRNAs. Dev Biol. 2004 Jun. 15; 270(2):488-98; Houbaviy H B, Murray M F, Sharp P A. Embryonic stem cell-specific MicroRNAs. Dev Cell. 2003 August; 5(2):351-8; USPTO Applications 20080025958, Cell-based RNA interference and related methods and compositions; 20070050146 MicroRNAs and uses thereof U.S. Pat. No. 7,232,806, MicroRNA molecules; WIPO 2007/054520, Methods for the identification of microRNAs and their application in research and human health).

In order to define the pluripotentcy of stem cells within and across phenotypes, the overriding aim must be to define and temporally stratify the factors and mechanisms controlling the differentiation of what really is a novel progenitor cell population heretofore uncharacterized, and unappreciated as a commercial clinical potential source of stem cells. As it will be “necessary to differentiate and expand these cells into specific cell lineages and in sufficient quantities”, the parent cell population should bear several inexorable characteristics in order to clinically and commercially viable, not the least of which are: 1—Have an initial population that does not impose undo isolation and expansion costs; 2—Have a relative temporal “stemness” that potentially permits the parent cells to undergo multiple differentiation pathways and thus achieve multiple phenotypes; 3—Be readily available without imposing untoward acquisition requirements; 4—Be expandable without loss of pluri-potency so that commercial operations are predictable and reproducible based on clinical demand; and 5—The expansion/differentiation processing should be free of imposing xenobiotic characters to the extent that there is no realized pathology imparted to the final clinical commercial tissue. We have now defined the signatures of microRNAs (miRs) in these cells as they exist in the stem cell state and in their differentiated normal counterpart, and that the modulation of miRs can produce the differentiation process of these stem cell precursors to altered phenotypic states, e.g. hematopoietic, pulmonary, or vascular phenotypes, and the utilization of miRs and antagomiRs as the differentiating agents, thus minimizing or even eliminating the need for xenobiotic materials and providing a defined commercial process that is dependent on the application of small synthetic molecules for production cycles.

We have now identified a source of adult mesenchymal stem cells that are highly homogeneous, provide >3×10⁷ cells upon initial isolation, can be cultured for >20 days without loss of initial stemness, and bear a relative temporal stemness at least twice as early as hematopoietic precursor cells, and, based on such discovery, are only marginally diminished in stemness from some embryonic cell lines. These cells bear numerous embryonic-like stem cell markers at levels paralleling those of many embryonic lines. Most importantly, we have now defined the specific microRNA profiles or signatures that define such cells as “stem cells” wherein this signature is validated by the use of proper anatomical control tissues and a span of other differentiated tissues.

### DESCRIPTION OF FIGURES

FIG. 1—Describes the potential of stem cell differentiation and the possible terminally differentiated phenotypes or tissue end stages. This figure specifically depicts the culturing of stem cells, their use in defining gene expression important to stem cell states, their use in drug or toxicity testing, and the induction of differentiation into terminal tissue stages such as muscle, bone, nerve & pancreatic tissues.

FIG. 2—Indicates the various layers of embryonic tissues and the tissues that arise from these layers; this figure provides a listing of the potential terminally differentiated phenotypes that may be produced from highly pluri-potent stem cells.

FIG. 3—Indicates human diseases that have immediate need for stem cells, stem cell products or synthetic stem cell signature materials with high pluripotent value and the potential to regulate disease progression or modify these disease states.

FIG. 4—Indicates the numerous embryonic stem cell markers and their relative significance in defining the degree of “stemness” as markers in class A or class B markers.

FIG. 5—Indicates the temporal expression of the Oct-3/4 gene product through 22 days of in vitro (IV) culturing of the Adult oral mesenchymal stem cells. This figure indicates that the significant expression of the embryonic stem cell marker Oct-3/4 is not lost throughout the culture process up to 22 days. The X axis represents the time in culture in days and the Y-axis represents the % positivity for the marker of interest (Oct-3/4).

FIG. 6—Indicates the temporal expression of the Tra1-60 gene product through 22 days of in vitro (IV) culturing of the adult oral mesenchymal stem cells. This figure indicates that the significant expression of the embryonic stem cell marker Tra1-60 is not lost throughout the culture process up to 22 days. The X axis represents the time in culture in days and the Y-axis represents the % positivity for the marker of interest (Tra1-60).

FIG. 7—Indicates the temporal expression of the SSEA-3 gene product through 22 days of in vitro (IV) culturing of the adult oral mesenchymal stem cells. This figure indicates that the significant expression of the embryonic stem cell marker SSEA-3 is not lost throughout the culture process up to 22 days. The X axis represents the time in culture in days and the Y-axis represents the % positivity for the marker of interest (SSEA-3).

FIG. 8—Indicates the temporal expression of the CD34 gene product through 22 days of in vitro (IV) culturing of the adult oral mesenchymal stem cells. This figure indicates that the significant expression of the blood stem cell marker CD34 is not realized by the oral mesenchymal stem cells throughout the culture process up to 22 days. The initial founding of this marker represents a blood cell carry-over from the biopsy process. The X axis represents the time in culture in days and the Y-axis represents the % positivity for the marker of interest (CD34).

FIG. 9—Indicates the temporal expression of the CD117 gene product C-kit/r) through 22 days of in vitro (IV) culturing of the adult oral mesenchymal stem cells. This figure indicates that the significant expression of the blood stem cell marker CD117 is not expressed by the oral mesenchymal stem cells throughout the culture process up to 22 days. The X axis represents the time in culture in days and the Y-axis represents the % positivity for the marker of interest (CD117).

FIG. 10—FIG. 10, a, b, and c represent microarray heat maps of the expression of specific microRNAs. Panel A represents the heat map data from the microRNA microarray of the adult oral mesenchymal stem cells (SC) in contrast to 27 other fully differentiated tissues. Note that the blue areas indicate lack of expression while the red areas represent highly expressed levels of specific microRNAs. Areas of substantial contrast are noted by boxing of the SC expression in contrast to the other tissues. It can be noted that FIG. 10B1 indicates an area of high expression of select microRNAs while FIGS. 10B2 and 10C3 represent areas of total suppression of microRNAs in the SC in contrast to the differentiated tissues. Specific microRNAs are listed for these areas and represent combinatorial targets for induction of differentiation along with other microRNAs.

### SUMMARY OF THE INVENTION

For the purposes of this application, the terms miR, miRNA and microRNA are used interchangeably.

For the purposes of this application, miRs are designated by their miR number and their corresponding ascention number where example data are presented. Where ascention numbers are not specifically included along side of the miR number, these ascention numbers and sequences are provided by reference in their entirety at http://microrna.sanger.ac.uk/. The ascention number is referenced to the structure and composition of the miR through the Sanger global data base, called miRBase, of miRs at http://microrna.sanger.ac.uk/. miRBase is the new home for microRNA data, incorporating the database and gene naming roles previously provided by the miRNA Registry, and including the new miRBase Target database.

miRBase contains three main sections:
miRBase Sequences contains all published miRNA sequences, genomic locations and associated annotation.
miRBase Targets is a newly developed database of predicted miRNA target genes.
miRBase Registry provides a confidential service assigning official names for novel miRNA genes prior to publication of their discovery.

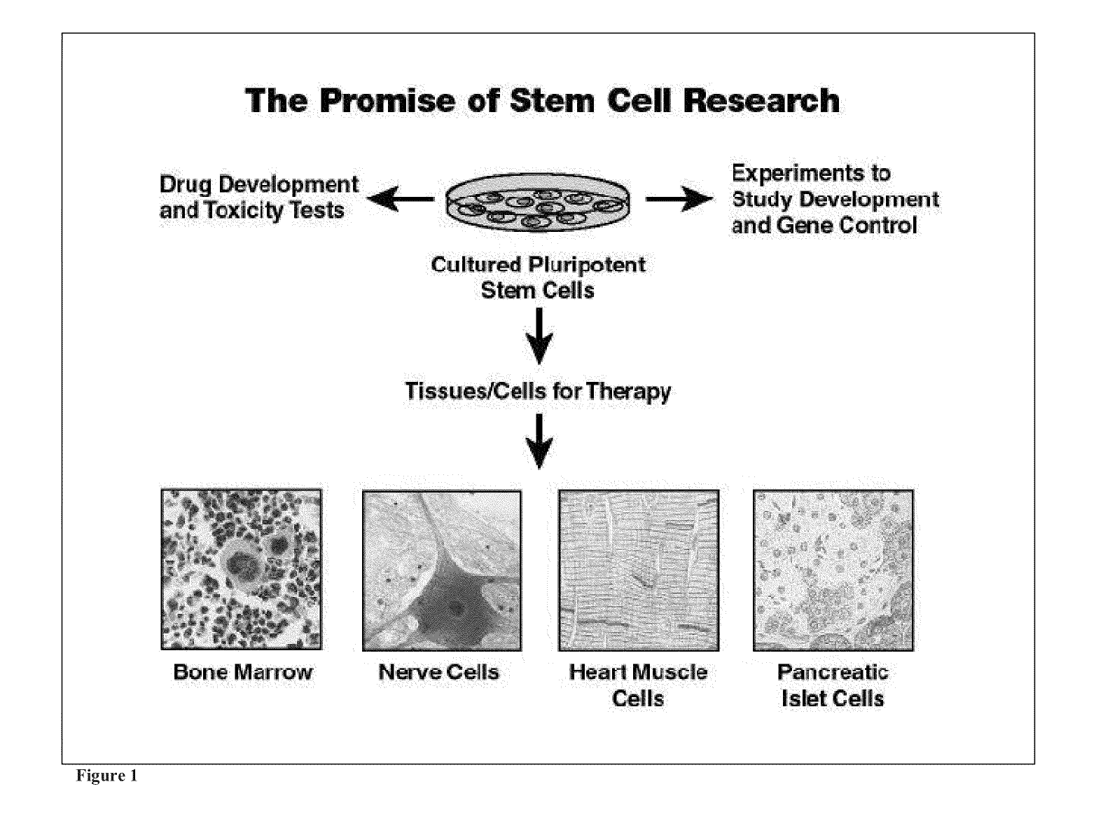

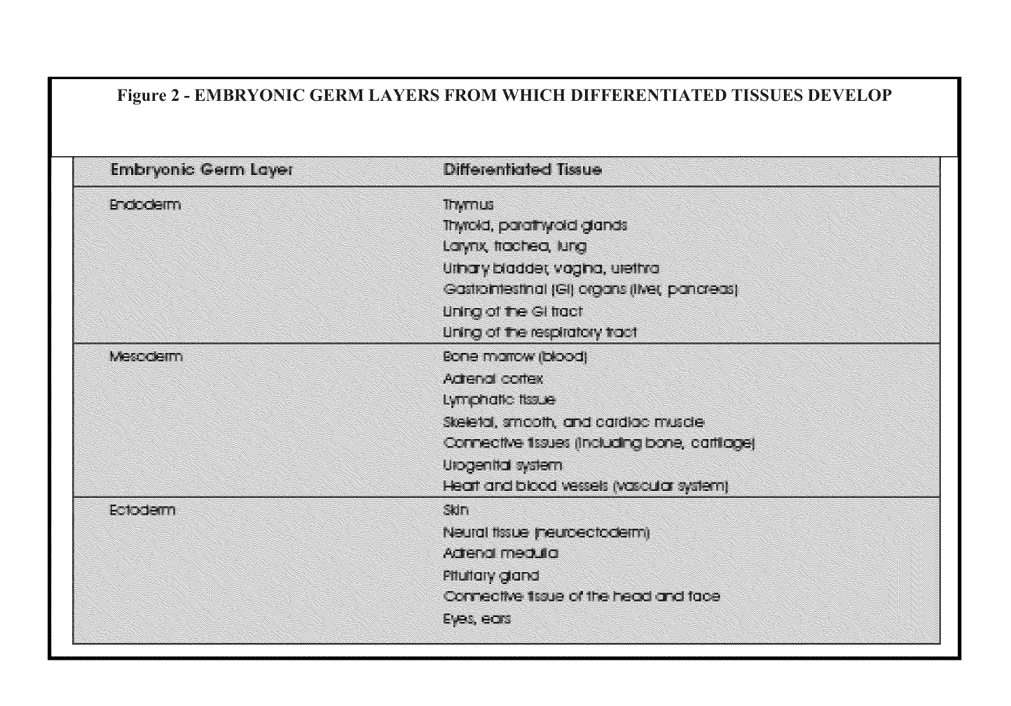

For the purposes of this application the terms “adult” and “stem cell”, unless designated as embryonic, refers to non-pre-perinatal mammals and adult stem cells derived from non-pre-perinatial, mammals, respectively, including human beings (Homo sapien sapien) and represent the pluripotent differentiation potential or greater of these stem cells as depicted in FIGS. 1 and 2 of this application.

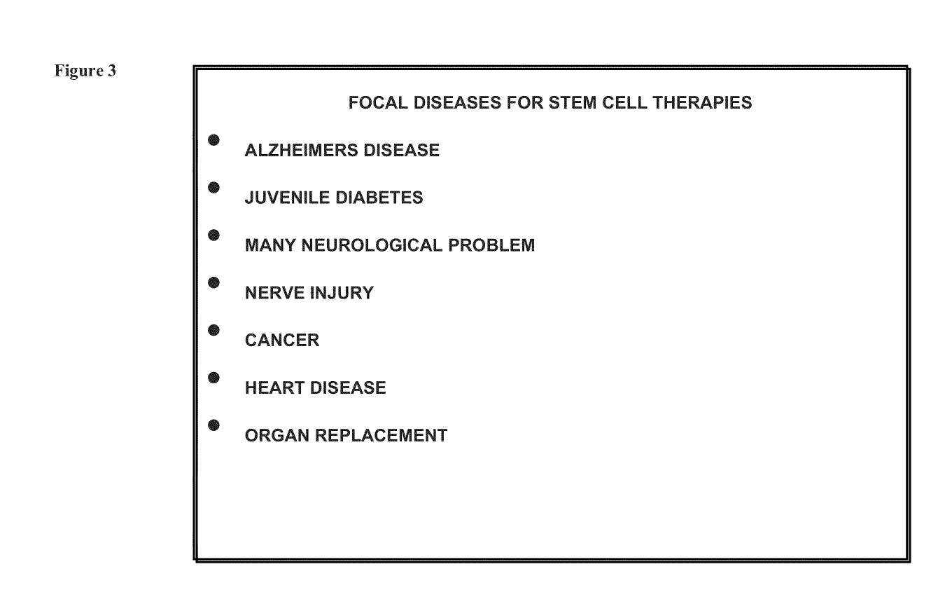

For the purposes of this application the therapeutic applications of stem cells, unless designated as embryonic, refers to adult stem cells derived from non-pre-perinatal mammals, including human beings, and represent the utility of these stem cells, either directly, or indirectly, in the treatment of diseases as outlined in FIG. 3 or other diseases which biologically or medically lend themselves to stem cell therapies or stem cell-derived therapies that utilizes such agents as microRNAs and stem cell-derived micro-RNA signatures as described in this application.

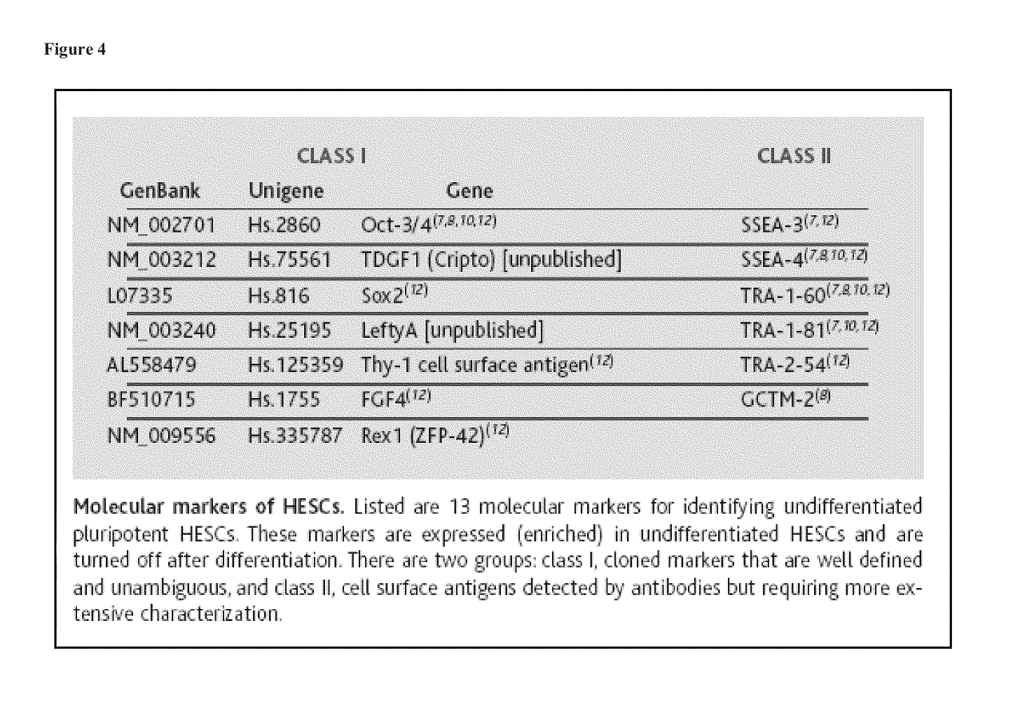

For the purposes of this application, the stem cell markers utilized in this application are derived from a consensus marker profile that defines pluripotentcy in the mammalian embryonic stem cell as these may be applied to non-hematological stem cells as outlined in FIG. 4 of this application. In certain embodiments, the stem cell markers are selected from the following group of markers: Oct3/4, SSEA1, SSEA3, SSEA4, Tra-1-60, TRA-1-81, SOX2, NANOG, CD44, CD34, CD9, CD133, CD117, CD4, CD8, MART, and CD24 well known to those skilled in the art of sciences and clinical approaches of embryonic stem cell investigations.

The present invention is based on the identification of stem cell specific miRNA signatures that are uniquely expressed in adult stem cells.

Accordingly, the invention encompasses a method of identifying such stem cells with near embryonic stem cell capability of a single rare stem cell up to clusters of even 20 million cells or greater. The method includes measuring the level of at least one miR gene product in a biological sample derived from the adult subject. An alteration in the level of the miR gene product in the biological sample as compared to the level of a corresponding miR gene product in an anatomically correct control sample, as indicative of the subject either bearing or not, clinically utilizable adult stem cells. The stem cell signature derived therefrom can in fact be utilized to define the presence or absence of rare stem cells in fully differentiated tissue and is thus a pan stem cell signature.

In certain embodiments, the miR gene product with altered expression is selected from the following group: hsa-mir-517b, hsa-mir-517c, hsa-mir-517a, hsa-mir-519a-1, hsa-mir-519a-2No1, hsa-mir-520gNo1, hsa-mir-135bNo1, hsa_mir_{—}95 right, hsa-mir-205-prec, hsa-mir-383No1, hsa-mir-022-prec, hsa-mir-509No1, hsa-mir-188-prec, hsa-mir-187-precNo1, hsa-mir-200cNo1, hsa-mir-222-precNo1, hsa-mir-211-precNo2, hsa-mir-519dNo1, hsa-mir-095-prec-4, hsa-mir-338No1, hsa-mir-009-3No2, hsa-mir-127-prec, hsa-mir-001b-1-prec1, hsa-mir-26a-2No1, hsa-mir-203-precNo1, hsa-mir-199b-precNo2, hsa-mir-029c-prec, hsa-mir-105-2No2, hsa-mir-326No2, hsa-mir-519b, hsa-mir-106-prec-X, hsa-mir-425No1, hsa-mir-105-2No1, hsa-mir-369No1, hsa-mir-132-precNo2, hsa-mir-181b-2No2, hsa-mir-030b-precNo1, hsa-mir-100No1, hsa-mir-026b-prec, hsa-mir-015b-precNo1, hsa-mir-345No2, hsa-mir-030a-precNo2, hsa-mir-020-prec, hsa-mir-328No1, hsa-mir-192-2/3No1, hsa-mir-149-prec, hsa-mir-30eNo2, hsa-mir-200bNo1, hsa-miR-126*No1, hsa-mir-486, hsa-mir-212-precNo1, hsa-mir-499No1, hsa-mir-337No1, hsa-mir-321No1, hsa-mir-139-prec, hsa-mir-125b-2-precNo2, hsa-mir-100-1/2-prec, hsa-mir-208-prec, hsa-mir-030c-prec, hsa-mir-126No2, hsa-mir-410No1, hsa-mir-450-1, hsa-mir-196-1-precNo2, hsa-mir-030a-precNo1, hsa-mir-184-precNo2, hsa-mir-030d-precNo2, hsa-mir-133bNo2, hsa-mir-30c-1No1, hsa-mir-361No1, hsa-mir-30c-2No1, hsa-mir-29b-1No1, hsa-mir-321No2, hsa-mir-192No1, hsa-mir-491No2, hsa-miR-373*No1, hsa_mir_{—}320_Hcd306 right, hsa-mir-335No1, hsa-mir-181c-precNo1, hsa-mir-500No2, hsa-mir-346No1, hsa-mir-485-3p, hsa-mir-155-prec, hsa-mir-30eNo1, hsa-mir-218-2-precNo2, hsa-mir-099-prec-21, hsa-mir-102-prec-1, hsa-mir-101-1/2-precNo1, hsa-mir-130bNo1, hsa-mir-422aNo2 or combinations thereof.

In one embodiment, the level of the gene product in the biological sample is greater than the level of its corresponding miR gene product in the control sample. Such under-expressed gene products include: hsa-mir-135bNo1, hsa_mir_{—}95 right, hsa-mir-211-precNo2, hsa-mir-095-prec-4, hsa-mir-338No1, hsa-mir-26a-2No1, hsa-mir-199b-precNo2, hsa-mir-029c-prec, hsa-mir-106-prec-X, hsa-mir-425No1, hsa-mir-105-2No1, hsa-mir-030b-precNo1, hsa-mir-100No1, hsa-mir-026b-prec, hsa-mir-030a-precNo2, hsa-mir-020-prec, hsa-mir-328No1, hsa-mir-149-prec, hsa-miR-126*No1, hsa-mir-212-precNo1, hsa-mir-100-1/2-prec, hsa-mir-030c-prec, hsa-mir-030a-precNo1, hsa-mir-30c-1No1, hsa-mir-30c-2No1, hsa-mir-491No2, hsa-mir-099-prec-21 or combinations thereof.

In one embodiment, the level of the gene product in the biological sample is less than the level of its corresponding miR gene product in the control sample. Such under-expressed gene products include: hsa-mir-517b, hsa-mir-517c, hsa-mir-517a, hsa-mir-519a-1, hsa-mir-519a-2No1, hsa-mir-520gNo1, hsa-mir-205-prec, hsa-mir-383No1, hsa-mir-022-prec, hsa-mir-509No1, hsa-mir-188-prec, hsa-mir-187-precNo1, hsa-mir-200cNo1, hsa-mir-222-precNo1, hsa-mir-519dNo1, hsa-mir-009-3No2, hsa-mir-127-prec, hsa-mir-001b-1-prec1, hsa-mir-203-precNo1, hsa-mir-326No2, hsa-mir-519b, hsa-mir-486, hsa-mir-321No1, hsa-mir-196-1-precNo2,

hsa-mir-321No or combinations thereof.

The invention also provides another method of determining whether a subject has available stem cell populations that may be accessible, isolated, and clinically utilized. The method includes: (a) providing a test sample from the adult subject's tissue wherein the test sample contains multiple miR gene products; (b) assaying the expression level of the miR gene products in the test sample to provide an miR expression profile for the test sample; (c) comparing the miR expression profile of the test sample to a corresponding miR expression profile generated from a control sample. A difference between the miR expression profile of the test sample and the miR expression profile of the control sample is indicative of the presence of stem cells.

In one embodiment, the multiple miR gene products correspond to a substantial portion of the full complement of miR genes in a cell. In other embodiments, the multiple miR gene products correspond to about 95%, 90%, 80%, 70% or 60% of the full complement of miR genes in a cell.

In another embodiment, the multiple miR gene products include one or more miR gene products selected from the group consisting of: hsa-mir-346-No.1, hsa_mir_{—}320_Hcd306 left, hsa-mir-320-No.2, hsa-mir-184-prec-No.2, hsa-mir-328-No.1, hsa-mir-130b-No.2, hsa-mir-196b-No.2, sa-mir-218-1-prec, hsa-mir-219-prec, hsa-mir-425-No.2, hsa-mir-107-No.1, hsa-mir-107-prec-No.10, hsa-mir-103-2-prec, hsa-mir-103-prec-5=103-1, hsa-mir-15a-No.1, hsa-mir-15a-2-prec-No.1, hsa-mir-15b-prec-No. 1, hsa-mir-451-No.1, hsa-mir-123-prec-No.2, hsa-mir-126*-No.2, hsa-mir-126-No. 1, hsa-mir-195-prec, hsa-mir-16-2-No.1, hsa-mir-16-1-No.1, hsa-mir-17-prec-No.1, hsa-mir-16a-chr13, hsa-mir-16b-chr3, hsa-mir-19a-prec, hsa-mir-194-prec-No.2, hsa-mir-342-No.2, hsa-mir-429-No.2, hsa-mir-218-2-prec-No.2, hsa-let7f-1-prec-No.1, hsa-mir-188-prec, hsa-mir-203-prec-No.1, hsa-mir-205-prec, hsa-mir-023a-prec, hsa-mir-023b-prec, hsa-mir-024-1-prec-No.1, hsa-mir-024-2-prec, hsa-mir-027a-prec, hsa-mir-027b-prec, hsa-mir-021-prec17-No.1, hsa-mir-021-No.1, hsa-mir-187-prec-No.1, hsa-mir-143-prec, hsa-mir-145-prec, hsa-mir-031-prec, hsa-mir-194-2-No.1, hsa-mir-215-prec-No.2, hsa-mir-192-2/3-No.1, hsa-mir-192-No.1, hsa-mir-215-prec-No.1, hsa-mir-375, hsa-mir-141-prec-No.1, hsa-mir-200a-prec, hsa-mir-200b-No.1, hsa-mir-192-/3-No.2, hsa-mir-370-No.2, hsa-mir-152-prec-No.1, hsa-mir-148b-No. 1, hsa-mir-148a-No.1, hsa-mir-148-prec, hsa-mir-206-prec-No.1, hsa-mir-001b-1-prec-No.1, hsa-mir-133a-1, hsa-mir-007-1-prec or combinations thereof.

The level of said miR gene product can be measured using a variety of techniques that are well known in the art. These techniques include but are not limited to amplification-based assays, hybridization-based assays, and microarray analyses. In other embodiments, the level of the miR gene product can be determined by measuring the corresponding miR gene copy in the sample.

The biological sample obtained from the adult subject can include any tissue, tissue extract, juice, exudate, secretion, or extraction from a post-natal individual. The analysis can be conducted in vitro on living cells, or on fixed, frozen or dead tissue.

The invention also contemplates a kit for elucidating adult stem cells in a subject or tissues or exudates, or juices, or samples of any character from a subject. Such a kit can include: (a) a means for measuring the level of at least one miR gene product in a biological sample derived from the subject's tissue, and (b) a means for comparing the level of the miR gene product in the biological sample to the level of a corresponding miR gene product in a control sample. A detected difference between the levels of the miR gene products in the biological sample as compared with the level of the corresponding miR gene products in an anatomically correct control sample defines signature component miRs.

The invention also provides a method of inhibiting utilizing miR signatures therapeutically, to derive clinically valuable tissues in vitro or application of synthetic miRs in vivo to modulate tissues for desired clinical outcomes. Such a method includes administering to the subject an effective amount of an active miR of miR inhibitor molecule that is capable of reducing the amount of the miR gene product in the target cells.

In one embodiment, the inhibitor molecule is administered as naked RNA, in conjunction with a delivery agent. In another embodiment, the inhibitor molecule is administered as a nucleic acid encoding the inhibitor molecule.

The invention also provides for a method of identifying a stem cell modulating agent. This method comprises the steps of: (a) determining the expression level of at least one miR gene product which is over-expressed in a biological sample containing stem cells, thereby generating data for a pre-test expression level of said miR gene product; (b) contacting the biological sample with a test agent; (c) determining the expression level of the miR gene product in the biological sample after step (b), thereby generating data for a post-test expression level; and (d) comparing the post-test expression level to the pre-test expression level of the miR gene product, wherein a decrease in the post-test expression level of the miR gene product is indicative that the test agent has stem cell modulating properties.

The invention also provides for another method of identifying an anti-stem cell agent, comprising the steps of (a) determining the expression level of at least one miR gene product which is under-expressed in a biological sample containing adult mesenchymal stem cells, thereby generating data for a pre-test level expression of said miR gene product; (b) contacting the biological sample with a test agent; (c) determining the expression level of the miR gene product in the biological sample, thereby generating data for a post-test level; and (d) comparing the post-test expression level to the pre-test expression level of said miR gene product, wherein an increase in the post-test expression level of the miR gene product is indicative that the test agent has anti-stem cell properties.

### DETAILED DESCRIPTION OF THE INVENTION

The invention will now be described with reference to more detailed examples. The examples illustrate how a person skilled in the art can make and use the invention, and are described here to provide enablement and best mode of the invention without imposing any limitations that are not recited in the claims.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein.

The undifferentiated state of embryonic stem cells is characterized by high level of expression of the POU transcription factor Octamer-3/4 (Oct-3/4). This relationship between Oct-3/4 and pluripotency has seen this transcription factor emerge as a marker of pluripotent stem cells. Undifferentiated human and murine pluripotent Embryonic Stem (ES) and Embryonic Carcinoma (EC) cells express Oct-3/4. Additionally, murine Embryonic Germ (EG) cells are also known to express Oct-3/4. Following stem cell differentiation, the level of Oct-3/4 expression is decreased. Pluripotent stem cells can also be characterized by the expression of a number of cell surface antigens. SSEA-1, a carbohydrate antigen, is a fucosylated derivative of type 2 polylactosamine and appears during late cleavage stages of mouse embryos. It is strongly expressed by undifferentiated, murine ES cells. Upon differentiation, murine ES cells are characterized by the loss of SSEA-1 expression and may be accompanied, in some instances, by the appearance of SSEA-3 and SSEA-4. In contrast, human ES and EC cells typically express SSEA-3 and SSEA-4 but not SSEA-1, while their differentiation is characterized by down regulation of SSEA-3 and SSEA-4 and an up regulation of SSEA-1. Undifferentiated, human ES cells also express the keratin sulphate-associated antigens, TRA-1-60 and TRA-1-81.

In some embodiments, potential stem cells, heretofore unrecognized as a potential major source of highly pluripotent stem cells, were extracted from the anatomical precursor of the third molar. This extraction was performed as a routine prophylaxis in young male and female peripubertal humans. We visualized embryonic stem cell markers in these tissues via immunohistochemistry (IHC), fluorescence immunohistochemistry (FIHC), and via other histochemistry known to all skilled in the art of pathology and histological analysis. The stem cell markers are selected from the following group of markers: Oct-3/4, SSEA1, SSEA3, SSEA4, Tra-1-60, TRA-1-81, SOX2, NANOG, CD44, CD34, CD9, CD133, CD117, CD4, CD8, MART, and CD24 well known to those skilled in the art of sciences and clinical approaches of embryonic stem cell investigations.

As used herein interchangeably, a “miR gene product,” “microRNA,” or “miRNA refers to the unprocessed or processed RNA transcript from an miR gene. The unprocessed miR gene transcript is also called a “miRNA precursor.” These miRNA precursors are converted to mature forms of miRNAs through a stepwise processing described earlier (Liu Y, Zheng Y, Ding G, Fang D, Zhang C, Bartold P M, Gronthos S, Shi S, Wang S. Periodontal Ligament Stem Cell-mediated Treatment for Periodontitis in Miniature Swine. Stem Cells. 2008 Jan. 31; [Epub PMID: 18238856]; Liu C G, Calin G A, Meloon B, Gamliel N, Sevignani C, Ferracin M, Dumitru C D, Shimizu M, Zupo S, Dono M, Alder H, Bullrich F, Negrini M, Croce C M. An oligonucleotide microchip for genome-wide microRNA profiling in human and mouse tissues. Proc Natl Acad Sci USA. 2004 Jun. 29; 101(26):9740-4). It is believed that the processing first generates (A) a large primary precursor, or a “pri-miRNA,” that is then processed by the nuclear enzyme Drosha to produce (B) a putative precursor, or a “pre-miRNA.” The pre-miRNA usually has 50-90 nucleotides (nt), particularly 60-80 nt. The terms “miRNA precursors” or “unprocessed miRNA used herein are inclusive of both the pri-miRNA and the pre-miRNA. An active 19-25 nt mature miRNA is processed from the pre-miRNA by the ribonuclease Dicer.

The active or mature miRNA molecule can be obtained from the miRNA precursor through natural processing routes (e.g., using intact cells or cell lysates) or by synthetic processing routes (e.g., using isolated processing enzymes, such as isolated Dicer, Argonaut, or RNAase III). It is understood that the active 19-25 nucleotide RNA molecule can also be produced directly by biological or chemical synthesis, without having been processed from the miR precursor.

Both the pri-miRNA and pre-miRNA molecules have a characteristic “hairpin sequence,” which is an oligonucleotide sequence having a first half which is at least partially complementary to a second half thereof, thereby causing the halves to fold onto themselves, forming a “hairpin structure.” The hairpin structure is typically made of a “stem” part, which consists of the complementary or partially complementary sequences, and a “loop” part, which is a region located between the two complementary strands of the stem.

As used herein, the term “miR gene expression” refers to the production of miR gene products from a miR gene, including processing of the miR precursor into a mature miRNA gene product.

The level of the target miR gene product is measured in a biological sample legally obtained from a subject. For example, a biological sample can be removed from a subject via surgery. Such a biological sample can include a tissue or cell biopsy. Alternatively, a biological sample can include tissue juice extracted after stimulation of an organ or tissue. In another example, a tissue sample may be removed from a cadaver or sourced by other means known in the medical arts. The tissue may be fresh, fresh frozen, frozen, fixed, e.g. fin formaldehyde, or paraffin, removed from the subject. A corresponding control sample can be obtained from unaffected tissues of the subject, from a normal subject or population of normal subjects. The control sample is then processed along with the test sample from the subject, so that the levels of miR gene product produced from a given miR gene in the subject's sample can be compared to the corresponding miR gene product levels from the control sample.

As used herein, the term “subject” includes any animal whose biological sample contains a miR gene product. The animal can be a mammal and can include pet animals, such as dogs and cats; farm animals, such as cows, horses and sheep; laboratory animals, such as rats, mice and rabbits; and primates, such as monkeys and humans. In the embodiments, the mammal is a human being (Homo sapien sapien).

An alteration (i.e., an increase or decrease) in the level of a miR gene product in the sample obtained from the subject, relative to the level of a corresponding miR gene product in a control sample, is indicative of the presence of stem cells in the subject. In some embodiments, the level of the target miR gene product in the test sample is greater than the level of the corresponding miR gene product in the control sample (i.e., expression of the miR gene product is “up-regulated” or the miR gene product is “over-expressed”). As used herein, expression of a miR gene product is “up-regulated” when the amount of miR gene product in a test sample from a subject is greater than the amount of the same gene product in a control sample.

In some embodiments, the up-regulated miR gene products include one or more of the following: hsa-mir-135bNo1, hsa_mir_{—}95 right, hsa-mir-211-precNo2, hsa-mir-095-prec-4, hsa-mir-338No1, hsa-mir-26a-2No1, hsa-mir-199b-precNo2, hsa-mir-029c-prec, hsa-mir-106-prec-X, hsa-mir-425No1, hsa-mir-105-2No1, hsa-mir-030b-precNo1, hsa-mir-100No1, hsa-mir-026b-prec, hsa-mir-030a-precNo2, hsa-mir-020-prec, hsa-mir-328No1, hsa-mir-149-prec, hsa-miR-126*No1, hsa-mir-212-precNo1, hsa-mir-100-1/2-prec, hsa-mir-030c-prec, hsa-mir-030a-precNo1, hsa-mir-30c-1No1, hsa-mir-30c-2No1, hsa-mir-491No2, hsa-mir-099-prec-21, hsa-mir-494-No.1, hsa-mir-346-No.1, hsa_mir_{—}320_Hcd306 left, hsa-mir-320-No.2, hsa-mir-184-prec-No.2, hsa-mir-328-No.1, hsa-mir-130b-No.2, hsa-mir-196b-No.2,

In other embodiments, the level of the target miR gene product in the test sample is less than the level of the corresponding miR gene product in the control sample (i.e., expression of the miR gene product is “down-regulated” or the miR gene product is “underexpressed”). As used herein, expression of a miR gene is “down-regulated” when the amount of miR gene product in a test sample from a subject is less than the amount of the same gene product in a control sample. The relative miR gene expression in the control samples can be determined with respect to one or more RNA expression standards. The standards can comprise, for example, the average level of miR gene expression previously obtained for a population of normal controls.

In some embodiments, the down-regulated miR gene products include one or more of the following: hsa-mir-517b, hsa-mir-517c, hsa-mir-517a, hsa-mir-519a-1, hsa-mir-519a-2No1, hsa-mir-520gNo1, hsa-mir-205-prec, hsa-mir-383No1, hsa-mir-022-prec, hsa-mir-509No1, hsa-mir-188-prec, hsa-mir-187-precNo1, hsa-mir-200cNo1, hsa-mir-222-precNo1, hsa-mir-519dNo1, hsa-mir-009-3No2, hsa-mir-127-prec, hsa-mir-001b-1-prec1, hsa-mir-203-precNo1, hsa-mir-326No2, hsa-mir-519b, hsa-mir-486, hsa-mir-321No1, hsa-mir-196-1-precNo2, hsa-mir-321 No2, hsa-mir-219-prec, hsa-mir-425-No.2, hsa-mir-107-No.1, hsa-mir-107-prec-No.10, hsa-mir-103-2-prec, hsa-mir-103-prec-5-103-1, hsa-mir-15a-No.1, hsa-mir-15a-2-prec-No.1, hsa-mir-15b-prec-No.1, hsa-mir-451-No.1, hsa-mir-123-prec-No.2, hsa-mir-126*-No.2, hsa-mir-126-No.1, hsa-mir-195-prec, hsa-mir-16-2-No.1, hsa-mir-16-1-No.1, hsa-mir-17-prec-No.1, hsa-mir-16a-chr13, hsa-mir-16b-chr3, hsa-mir-19a-prec, hsa-mir-194-prec-No.2, hsa-mir-342-No.2, hsa-mir-429-No.2, hsa-mir-218-2-prec-No.2, hsa-let 7f-1-prec-No.1, hsa-mir-188-prec, hsa-mir-203-prec-No.1, hsa-mir-205-prec, hsa-mir-023a-prec, hsa-mir-023b-prec, hsa-mir-024-1-prec-No.1, hsa-mir-024-2-prec, hsa-mir-027a-prec, hsa-mir-027b-prec, hsa-mir-021-prec17-No.1, hsa-mir-021-No.1, hsa-mir-187-prec-No.1, hsa-mir-143-prec, hsa-mir-145-prec, hsa-mir-031-prec, hsa-mir-194-2-No.1, hsa-mir-215-prec-No.2, hsa-mir-192-2/3-No.1, hsa-mir-192-No.1, hsa-mir-215-prec-No.1, hsa-mir-375, hsa-mir-141-prec-No.1, hsa-mir-200a-prec, hsa-mir-200b-No.1, hsa-mir-192-/3-No.2, hsa-mir-370-No.2, hsa-mir-152-prec-No.1, hsa-mir-148b-No.1, hsa-mir-148a-No.1, hsa-mir-148-prec, hsa-mir-206-prec-No.1, hsa-mir-001b-1-prec-No.1, hsa-mir-133a-1, hsa-mir-007-1-prec or combinations thereof.

Since more than one miR gene products is associated with stem cell “stemness”, it is understood that stem cell identification can be diagnosed by evaluating any one of the listed miR gene products, or by evaluating any combination of the listed miR gene products that when profiled, are indicative of the presence of stem cells. In some examples, a miR gene product that is uniquely associated with stem cells is evaluated.

A change in levels of miR gene products associated with stem cells can be detected prior to, or in the early stages of, the development of such cells of a subject. The invention therefore also provides a method for screening a subject or material derived from a subject, comprising evaluating the level of at least one miR gene product, or a combination of miR gene products, associated with stem cell elucidation in a biological sample obtained from the subject. Accordingly, an alteration in the level of the miR gene product, or combination of miR gene products, in the biological sample as compared to the level of a corresponding miR gene product in a control sample, is indicative of the subject bearing stem cells. The biological sample used for such screening can include tissue that is either normal or suspected of any disease. Subjects with a change in the level of one or more miR gene products associated with stem cell signature(s) are candidates for further development of monitoring and/or testing and/or derivation of stem cells. Such further testing can comprise histological examination of tissue samples, or other techniques within the skill in the art.

The term “target nucleotide sequence” or “target nucleotide” as used herein, refers to the polynucleotide sequence that is sought to be detected. Target nucleotide sequence is intended to include DNA (e.g., cDNA or genomic DNA), RNA, analogs of the DNA or RNA generated using nucleotide analogs, and derivatives, fragments and homologs thereof.

The level of a miR gene product in a sample can be measured using any technique that is suitable for detecting RNA expression levels in a biological sample. Suitable techniques for determining RNA expression levels in biological sample include amplification-based and hybridization-based assays.

Amplification-based assays use a nucleic acid sequence of the miR gene product, or the miR gene, as a template in an amplification reaction (for example polymerase chain reaction or PCR). The relative number of miR gene transcripts can also be determined by reverse transcription of miR gene transcripts, followed by amplification of the reverse-transcribed transcripts by polymerase chain reaction (RT-PCR). The levels of miR gene transcripts can be quantified in comparison with an internal standard, for example, the level of mRNA from a “housekeeping” gene present in the same sample. A suitable “housekeeping” gene for use as an internal standard includes, e.g., 18s rRNA, myosin or glyceraldehyde-3-phosphate dehydrogenase (G3PDH). In a quantitative amplification, the amount of amplification product will be proportional to the amount of template in the original sample. Methods of real-time quantitative PCR or RT-PCR using TaqMan probes are well known in the art. Other examples of amplification-based assays for detection of miRNAs are well known in the art.

Hybridization-based assays can also be used to detect the level of miR gene products in a sample. These assays, including for example Northern blot analysis, in-situ hybridization, solution hybridization, and RNAse protection assay, RNase protection assay, are well known to those of skill in the art.

As used herein, the term “hybridization” refers to the complementary base-pairing interaction of one nucleic acid with another nucleic acid that results in formation of a duplex, triplex, or other higher-ordered structure, and is used herein interchangeably with “annealing.” Typically, the primary interaction is base specific, e.g., A/T and G/C, by Watson/Crick and Hoogsteen-type hydrogen bonding. Base-stacking and hydrophobic interactions can also contribute to duplex stability. Conditions for hybridizing detector probes and primers to complementary and substantially complementary target sequences are well known. In general, whether such annealing takes place is influenced by, among other things, the length of the polynucleotides and the complementary, the pH, the temperature, the presence of mono- and divalent cations, the proportion of G and C nucleotides in the hybridizing region, the viscosity of the medium, and the presence of denaturants. Such variables influence the time required for hybridization. Thus, the preferred annealing conditions will depend upon the particular application. Such conditions, however, can be routinely determined by the person of ordinary skill in the art without undue experimentation. It will be appreciated that complementarity need not be perfect; there can be a small number of base pair mismatches that will minimally interfere with hybridization between the target sequence and the single stranded nucleic acids of the present teachings. However, if the number of base pair mismatches is so great that no hybridization can occur under minimally stringent conditions then the sequence is generally not a complementary target sequence. Thus, complementarity herein is meant that the probes or primers are sufficiently complementary to the target sequence to hybridize under the selected reaction conditions to achieve the ends of the present teachings.

A suitable technique for determining the level of RNA transcripts of a particular gene in a biological sample is Northern blotting. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question. (See Molecular Cloning: A Laboratory Manual, J. Sambrook et al., eds., 2nd edition, Cold Spring Harbor Laboratory Press, 1989, Chapter 7.)

Suitable probes for Northern blot hybridization of a given miR gene product can be produced from the nucleic acid sequences derived from the Sanger database, or other published sequences of known miRNA species that are available, for example on the miRNA registry at: http://www.sanger.ac.uk/Software/Rfam/mirna/index.shtml.

Methods for preparation of labeled DNA and RNA probes, and the conditions for hybridization thereof to target nucleotide sequences, are known in the art and are described in Molecular Cloning: A Laboratory Manual, J. Sambrook et al., eds., 2nd edition, Cold Spring Harbor Laboratory Press, 1989, Chapter 7.

A suitable technique for simultaneously measuring the expression level of multiple miR gene products in a sample is a high-throughput, microarray-based method. Such a technique may be used to, for example, determine the expression level of the transcripts of all known miR genes correlated with cancer. Such a method involves constructing an oligo library, in microchip format (i.e., a microarray), that contains a set of probe oligonucleotides that are specific for a set of miR genes. Using such a microarray, the expression level of multiple microRNAs in a biological sample is determined by reverse transcribing the RNAs to generate a set of target oligonucleotides, and hybridizing them to probe oligonucleotides on the microarray to generate a hybridization, or expression, profile. The hybridization profile of the test sample can then be compared to that of a control sample to determine which microRNAs have an altered expression level in cancer or precancerous cells. In one example, the oligolibrary contains probes corresponding to all known miRs from the human genome. The microarray may be expanded to include additional miRNAs as they are discovered. The array can contain two different oligonucletode probes for each miRNA, one containing the active sequence of the mature miR and the other being specific for the miR precursor. The array may also contain controls for hybridization stringency conditions. An example of a microarray technique for detecting miRNAs is described in Liu C G, Calin G A, Meloon B, Gamliel N, Sevignani C, Ferracin M, Dumitru C D, Shimizu M, Zupo S, Dono M, Alder H, Bullrich F, Negrini M, Croce C M. An oligonucleotide microchip for genome-wide microRNA profiling in human and mouse tissues. Proc Natl Acad Sci USA. 2004 Jun. 29; 101(26):9740-4; Volinia S, Calin G A, Liu C G, Ambs S, Cimmino A, Petrocca F, Visone R, Iorio M, Roldo C, Ferracin M, Prueitt R L, Yanaihara N, Lanza G, Scarpa A, Vecchione A, Negrini M, Harris C C, Croce C M. A microRNA expression signature of human solid tumors defines cancer gene targets. Proc Natl Acad Sci USA. 2006 Feb. 14; 103(7):2257-61.

Any of a number of hybridization-based assays can be used to detect the copy number of a miR gene in the cells of a biological sample. One such method is Southern blot analysis (Molecular Cloning: A Laboratory Manual, J. Sambrook et al., eds., 2nd edition, Cold Spring Harbor Laboratory Press, 1989, Chapter 7) where the genomic DNA is typically fragmented, separated electrophoretically, transferred to a membrane, and subsequently hybridized to a miR gene specific probe. Comparison of the intensity of the hybridization signal from the probe for the target region with a signal from a control probe from a region of normal nonamplified, single-copied genomic DNA in the same genome provides an estimate of the relative miR gene copy number, corresponding to the specific probe used. An increased signal compared to control represents the presence of amplification.

Amplification-based assays also can be used to measure the copy number of the miR gene. In such assays, the corresponding miR nucleic acid sequences act as a template in an amplification reaction (for example, PCR). In a quantitative amplification, the amount of amplification product will be proportional to the amount of template in the original sample. Comparison to appropriate controls provides a measure of the copy number of the miR gene, corresponding to the specific probe used, according to the principles discussed above. Methods of real-time quantitative PCR using TaqMan probes are well known in the art.

A TaqMan-based assay also can be used to quantify miR polynucleotides. TaqMan-based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymerase, for example, AmpliTaq, results in the cleavage of the TaqMan probe. This cleavage separates the 5′ fluorescent dye and the 3′ quenching agent, thereby resulting in an increase in fluorescence as a function of amplification (see, for example, http://www2.perkin-elmer.com).

Other examples of suitable amplification methods include, but are not limited to, ligase chain reaction (LCR), transcription amplification, self-sustained sequence replication, dot PCR, and linker adapter PCR, all well known in the genetic sciences arts.

One powerful method for determining DNA copy numbers uses microarray-based platforms. Microarray technology may be used because it offers high resolution. For example, the traditional CGH generally has a 20 Mb limited mapping resolution; whereas in microarray-based CGH, the fluorescence ratios of the differentially labeled test and reference genomic DNAs provide a locus-by-locus measure of DNA copy-number variation, thereby achieving increased mapping resolution. Details of various microarray methods can be found in the literature (Krützfeldt J, Rajewsky N, Braich R, Rajeev K G, Tuschl T, Manoharan M, Stoffel M. Silencing of microRNAs in vivo with ‘antagomirs’. Nature. 2005 Dec. 1; 438(7068):685-9; Liu C G, Calin G A, Meloon B, Gamliel N, Sevignani C, Ferracin M, Dumitru C D, Shimizu M, Zupo S, Dono M, Alder H, Bullrich F, Negrini M, Croce C M. An oligonucleotide microchip for genome-wide microRNA profiling in human and mouse tissues. Proc Natl Acad Sci USA. 2004 Jun. 29; 101(26): 9740-4.).

As used herein, “probe oligonucleotide” refers to an oligonucleotide that is capable of hybridizing to a target oligonucleotide. “Target oligonucleotide” refers to a molecule or sequence to be detected (for example, via hybridization) by or “probe oligonucleotide” specific for a miR that has a sequence selected to hybridize to a specific miR gene product, or to a reverse transcript of the specific miR gene product.

A stem cell marker profile and/or “expression profile”, and/or a “hybridization profile” of a particular stem cell sample is essentially a fingerprint of the state of the sample; while two states may have any particular gene similarly expressed, the evaluation of a number of genes simultaneously allows the generation of a gene expression profile that is unique to the state of the cell. That is, differentiated tissue or pathological tissue may be distinguished from stem cell tissue, benign tissue obtained from a part of a subject's anatomy. By comparing expression profiles of tissue in different states, information regarding which genes are important (including both up- and down-regulation of genes) in each of these states is obtained. The identification of sequences that are differentially expressed in stem cell tissue or differentiated tissue or pathological tissue, as well as differential expression resulting in different prognostic outcomes, allows the use of this information in a number of ways. For example, a particular treatment regime may be evaluated. Similarly, diagnosis may be done or confirmed by comparing patient samples with the known expression profiles. Furthermore, these gene expression profiles (or individual genes) allow screening of miR drug candidates that suppress or correct pathological states based on stem cell signature components or combinations thereof.

Accordingly, the target nucleotide sequence of the miR gene product to be detected can be: (a) a portion of, or the entire sequence of the mature miRNA; (b) a portion of, or the entire hairpin sequence of the miRNA precursor; or (c) a portion of or the entire sequence of the pri-miRNA; (d) the complement of sequences (a)-(c); or (d) a sequence that is substantially identical to the sequences (a)-(d). A substantially identical nucleic acid may have greater than 80%, 85%, 90%, 95%, 97%, 98% or 99% sequence identity to the target sequence (http://microrna.sanger.ac.uk).

In one example of the invention, the level of miR gene products is detected by profiling miRNA precursors on biopsy specimens of human stem cells. The sequences of those profiled miR gene products are provided at http://microrna.sanger.ac.uk for some 673 human mir genes or derivatives there from and are indicated with the precursor sequences (prefix=MI) and mature sequences (prefix=MIMAT). All nucleic acid sequences herein are given in the 5′ to 3′ direction. In another example, the level of miR gene products is detected by mircoarray miRNA on biopsy specimens of human stem cells and or their anatomically correct counterpart controls.

Also contemplated are a series of kits for evaluation of stem cell presence, in a subject's tissue to the extent that miR stem cell signatures may be utilized to provide therapeutic support in pathological tissue. Also indicated is the use of the kit to elucidate the presence of rare stem cells in all tissue where the miR signatures or combinations of miRs may be applied to stimulate the outgrowth of these rare cells where this may be desirable in pathological conditions. Such a kit can include: (a) a means for measuring the level of at least one miR gene product in a biological sample derived from the subject's tissue, and (b) a means for comparing the level of the miR gene product in the biological sample to the level of a corresponding miR gene product in a control sample. Accordingly, a detected difference between the level of the miR gene product in the biological sample as compared with the level of the corresponding miR gene product in the control sample is indicative of the subject either having, or being at risk for, specific pathological conditions. The combinations of miRs selected from the signature may depend on the over or under expression of the specific miR and/or the expression of the stem cell markers.

As used herein, an “effective amount” of an isolated miR gene product is an amount sufficient to inhibit progression of cancer in a patient suffering from cancer. One skilled in the art can readily determine an effective amount of an miR gene product to be administered to a given subject, by taking into account factors, such as the size and weight of the subject; the extent of disease penetration; the age, health and sex of the subject; the route of administration; and whether the administration is regional or systemic.

For example, an effective amount of an isolated miR gene product can be based on the level of pathological condition to be treated. The approximate mass can be determined by calculating the approximate volume of the mass, wherein one cubic centimeter of volume is roughly equivalent to one gram. An effective amount of the isolated miR gene product based on the weight of a tumor mass can be in the range of about 10-500 micrograms/gram of tissue mass. In certain embodiments, the tissue mass can be at least about 10 micrograms/gram of tissue mass, at least about 60 micrograms/gram of tissue mass or at least about 100 micrograms/gram of tissue mass.

An effective amount of an isolated miR gene product can also be based on the approximate or estimated body weight of a subject to be treated. Such effective amounts can be administered parenterally or enterally, as described herein and is typically based on dosage scale up form animal studies contrasting the wt^{0.75} (kg) of the animal/dose (some metric weight) to alleviate the target condition vs wt^{0.75} (kg) of the human subject/dose required where one solves for the dose required=x (as metric weight).

One skilled in the art can also readily determine an appropriate dosage regimen for the administration of an isolated miR gene product to a given subject. For example, a miR gene product can be administered to the subject once (e.g., as a single injection or deposition). Alternatively, a miR gene product can be administered once or twice daily to a subject for a period of days to several months, particularly from about three to about twenty-eight days, more particularly from about seven to about ten days. In a particular dosage regimen, a miR gene product is administered once a day for seven days. Where a dosage regimen comprises multiple administrations, it is understood that the effective amount of the miR gene product administered to the subject can comprise the total amount of gene product administered over the entire dosage regimen.

As used herein, an “isolated” miR gene product is one which is synthesized, or altered or removed from the natural state through human intervention. For example, a synthetic miR gene product, or a miR gene product partially or completely separated from the coexisting materials of its natural state, is considered to be “isolated.” An isolated miR gene product can exist in substantially-purified form, or can exist in a cell into which the miR gene product has been delivered.

The isolated gene products can be oligonucleotides comprising the functional mature miR gene product, oligonucleotides comprising the short hairpin of miRNA precursors containing the looped portion of the hairpin, duplex miRNA precursors lacking the hairpin, or vectors expressing such molecules. Thus, a miR gene product which is deliberately delivered to, or expressed in, a cell is considered an “isolated” miR gene product. A miR gene product produced inside a cell from a miR precursor molecule is also considered to be an “isolated” molecule.

In some embodiments, the isolated miR gene products include one or more of the following: hsa-mir-494-No.1, hsa-mir-346-No.1, hsa_mir_{—}320_Hcd306 left, hsa-mir-320-No.2, hsa-mir-184-prec-No.2, hsa-mir-328-No.1, hsa-mir-130b-No.2, hsa-mir-196b-No.2, hsa-mir-218-1-prec, hsa-mir-219-prec, hsa-mir-425-No.2, hsa-mir-107-No. 1, hsa-mir-107-prec-No.10, hsa-mir-103-2-prec, hsa-mir-103-prec-5=103-1, hsa-mir-15a-No.1, hsa-mir-15^{a}-2-prec-No.1, hsa-mir-15b-prec-No.1, hsa-mir-451-No.1, hsa-mir-123-prec-No.2, hsa-mir-126*-No.2, hsa-mir-126-No.1, hsa-mir-195-prec, hsa-mir-16-2-No.1, hsa-mir-16-1-No.1, hsa-mir-17-prec-No.1, hsa-mir-16a-chr13, hsa-mir-16b-chr3, hsa-mir-19a-prec, hsa-mir-194-prec-No.2, hsa-mir-342-No.2, hsa-mir-429-No.2, hsa-mir-218-2-prec-No.2, hsa-let 7f-1-prec-No.1, hsa-mir-188-prec, hsa-mir-203-prec-No.1, hsa-mir-205-prec, hsa-mir-023a-prec, hsa-mir-023b-prec, hsa-mir-024-1-prec-No.1, hsa-mir-024-2-prec, hsa-mir-027a-prec, hsa-mir-027b-prec, hsa-mir-021-prec17-No.1, hsa-mir-021-No.1, hsa-mir-187-prec-No.1, hsa-mir-143-prec, hsa-mir-145-prec, hsa-mir-031-prec, hsa-mir-194-2-No.1, hsa-mir-215-prec-No.2, hsa-mir-192-2/3-No. 1, hsa-mir-192-No.1, hsa-mir-215-prec-No.1, hsa-mir-375, hsa-mir-141-prec-No.1, hsa-mir-200a-prec, hsa-mir-200b-No.1, hsa-mir-192-/3-No.2, hsa-mir-370-No.2, hsa-mir-152-prec-No.1, hsa-mir-148b-No.1, hsa-mir-148a-No.1, hsa-mir-148-prec, hsa-mir-001b-1-prec-No.1, hsa-mir-133a-1, hsa-mir-007-1-prec or combinations thereof.

In some embodiments, the isolated miR gene products include one or more of the following: hsa-mir-517b, hsa-mir-517c, hsa-mir-517a, hsa-mir-519a-1, hsa-mir-519a-2No1, hsa-mir-520gNo1, hsa-mir-135bNo1, hsa_mir_{—}95 right, hsa-mir-205-prec, hsa-mir-383No1, hsa-mir-022-prec, hsa-mir-509No1, hsa-mir-188-prec, hsa-mir-187-precNo1, hsa-mir-200cNo1, hsa-mir-222-precNo1, hsa-mir-211-precNo2, hsa-mir-519dNo1, hsa-mir-095-prec-4, hsa-mir-338No1, hsa-mir-009-3No2, hsa-mir-127-prec, hsa-mir-001b-1-prec1, hsa-mir-26a-2No1, hsa-mir-203-precNo1, hsa-mir-199b-precNo2, hsa-mir-029c-prec, hsa-mir-105-2No2, hsa-mir-326No2, hsa-mir-519b, hsa-mir-106-prec-X, hsa-mir-425No1, hsa-mir-105-2No1, hsa-mir-369No1, hsa-mir-132-precNo2, hsa-mir-181b-2No2, hsa-mir-030b-precNo1, hsa-mir-100No1, hsa-mir-026b-prec, hsa-mir-015b-precNo1, hsa-mir-345No2, hsa-mir-030a-precNo2, hsa-mir-020-prec, hsa-mir-328No1, hsa-mir-192-2/3No1, hsa-mir-149-prec, hsa-mir-30eNo2, hsa-mir-200bNo1, hsa-miR-126*No1, hsa-mir-486, hsa-mir-212-precNo1, hsa-mir-499No1, hsa-mir-337No1, hsa-mir-321No1, hsa-mir-139-prec, hsa-mir-125b-2-precNo2, hsa-mir-100-1/2-prec, hsa-mir-208-prec, hsa-mir-030c-prec, hsa-mir-126No2, hsa-mir-410No1, hsa-mir-450-1, hsa-mir-196-1-precNo2, hsa-mir-030a-precNo1, hsa-mir-184-precNo2, hsa-mir-030d-precNo2, hsa-mir-133bNo2, hsa-mir-30c-1No1, hsa-mir-361No1, hsa-mir-30c-2No1, hsa-mir-29b-1No1, hsa-mir-321No2, hsa-mir-192No1, hsa-mir-491No2, hsa-miR-373*No1, hsa_mir_{—}320_Hcd306 right, hsa-mir-335No1, hsa-mir-181c-precNo1, hsa-mir-500No2, hsa-mir-346No1, hsa-mir-485-3p, hsa-mir-155-prec, hsa-mir-30eNo1, hsa-mir-218-2-precNo2, hsa-mir-099-prec-21, hsa-mir-102-prec-1, hsa-mir-101-1/2-precNo1, hsa-mir-130bNo1, hsa-mir-422aNo2, hsa-mir-330No2, hsa-mir-010a-precNo1, hsa-mir-017-precNo2, hsa-mir-494No1, hsa-mir-26a-1No1, hsa-mir-130bNo2, hsa-mir-034-prec No2, hsa-mir-345No1, hsa-mir-219-1No1, hsa-mir-374No1, hsa-mir-148aNo1, hsa-mir-373No2, hsa-mir-106aNo1, hsa-mir-517b, hsa-mir-517c, hsa-mir-517a, hsa-mir-519a-1, hsa-mir-519a-2 No1, hsa-mir-520gNo1, hsa-mir-135bNo1, hsa_mir_{—}95 right, hsa-mir-205-prec, hsa-mir-383 No1, hsa-mir-022-prec, hsa-mir-509No1, hsa-mir-188-prec, hsa-mir-187-precNo1, hsa-mir-200c No1, hsa-mir-222-precNo1, hsa-mir-211-precNo2, hsa-mir-519dNo1, hsa-mir-095-prec-4, hsa-mir-338No1, hsa-mir-009-3No2, hsa-mir-127-prec, hsa-mir-001b-1-prec1, hsa-mir-26a-2No1, hsa-mir-203-precNo1, hsa-mir-199b-precNo2, hsa-mir-029c-prec, hsa-mir-105-2No2, hsa-mir-326No2, hsa-mir-519b, hsa-mir-106-prec-X, hsa-mir-425No1, hsa-mir-105-2No1, hsa-mir-369 No1, hsa-mir-132-precNo2, hsa-mir-181b-2No2, hsa-mir-030b-precNo1, hsa-mir-100No1, hsa-mir-026b-prec, hsa-mir-015b-precNo1, hsa-mir-345No2, hsa-mir-030a-precNo2, hsa-mir-020-prec, hsa-mir-328No1, hsa-mir-192-2/3No1, hsa-mir-149-prec, hsa-mir-30eNo2, hsa-mir-200bNo1, hsa-miR-126*No1, hsa-mir-486, hsa-mir-212-precNo1, hsa-mir-499No1, hsa-mir-337No1, hsa-mir-321No1, hsa-mir-139-prec, hsa-mir-125b-2-precNo2, hsa-mir-100-1/2-prec, hsa-mir-208-prec, hsa-mir-030c-prec, hsa-mir-126No2, hsa-mir-410No1, hsa-mir-450-1, hsa-mir-196-1-precNo2, hsa-mir-030a-precNo1, hsa-mir-184-precNo2, hsa-mir-030d-precNo2, hsa-mir-133bNo2, hsa-mir-30c-1No1, hsa-mir-361No1, hsa-mir-30c-2No1, hsa-mir-29b-1No1, hsa-mir-321No2, hsa-mir-192No1, hsa-mir-491No2, hsa-miR-373*No1, hsa_mir_{—}320_Hcd306 right, hsa-mir-335No1, hsa-mir-181c-precNo1, hsa-mir-500No2, hsa-mir-346No1, hsa-mir-485-3p, hsa-mir-155-prec, hsa-mir-30eNo1, hsa-mir-218-2-precNo2, hsa-mir-099-prec-21, hsa-mir-102-prec-1, hsa-mir-101-1/2-precNo1, hsa-mir-130bNo1, hsa-mir-422a No2 or combinations thereof.

Isolated miR gene products can be obtained using a number of standard techniques. For example, the miR gene products can be chemically synthesized or recombinantly produced using methods known in the art. In one embodiment, miR gene products are chemically synthesized using appropriately protected ribonucleoside phosphoramidites and a conventional DNA/RNA synthesizer. Commercial suppliers of synthetic RNA molecules or synthesis reagents include, e.g., Proligo (Hamburg, Germany), Dharmacon Research (Lafayette, Colo., U.S.A.), Pierce Chemical (part of Perbio Science, Rockford, Ill., U.S.A.), Glen Research (Sterling, Va., U.S.A.), Chem Genes (Ashland, Mass., U.S.A.) and Cruachem (Glasgow, UK).

Any viral vector capable of accepting the coding sequences for the miR gene products can be used; for example, vectors derived from adenovirus (AV); adeno-associated virus (AAV); retroviruses (e.g., lentiviruses (LV), Rhabdoviruses, murine leukemia virus); herpes virus, and the like. The tropism of the viral vectors can be modified by pseudotyping the vectors with envelope proteins or other surface antigens from other viruses, or by substituting different viral capsid proteins, as appropriate.

In other embodiments of the treatment methods of the invention, an effective amount of at least one compound which inhibits miR expression can also be administered to the subject. As used herein, “inhibiting miR expression” means that the production of the active, mature form of miR gene product after treatment is less than the amount produced prior to treatment. One skilled in the art can readily determine whether miR expression has been inhibited in a target cell, using for example the techniques for determining miR transcript level discussed above for the diagnostic method. Inhibition can occur at the level of gene expression (i.e., by inhibiting transcription of a miR gene encoding the miR gene product) or at the level of processing (e.g., by inhibiting processing of a miR precursor into a mature, active miR).

For example, an effective amount of the expression-inhibiting compound can be based on the approximate weight of a target tissue mass to be treated. The approximate weight of a tissue mass can be determined by calculating the approximate volume of the mass, wherein one cubic centimeter of volume is roughly equivalent to one gram. An effective amount based on the weight of a tissue mass can be between about 10-500 micrograms/gram of tissue mass, at least about 10 micrograms/gram of tissue mass, at least about 60 micrograms/gram of tissue mass, and at least about 100 micrograms/gram of tissue mass.

An effective amount of a compound that inhibits miR expression can also be based on the approximate or estimated body weight of a subject to be treated. Such effective amounts are administered parenterally or enterally, among others, as described herein. For example, an effective amount of the expression-inhibiting compound administered to a subject can range from about 5-3000 micrograms/kg of body weight, from about 700-1000 micrograms/kg of body weight, or it can be greater than about 1000 micrograms/kg of body weight.

One skilled in the art can also readily determine an appropriate dosage regimen for administering a compound that inhibits miR expression to a given subject. For example, an expression-inhibiting compound can be administered to the subject once (e.g., as a single injection or deposition). Alternatively, an expression-inhibiting compound can be administered once or twice daily to a subject for a period of from about a few days to a few months, from about three to about twenty-eight days, or from about seven to about ten days. In a particular dosage regimen, an expression-inhibiting compound is administered once a day for seven days. Where a dosage regimen comprises multiple administrations, it is understood that the effective amount of the expression-inhibiting compound administered to the subject can comprise the total amount of compound administered over the entire dosage regimen.

In some embodiments, the miR gene products whose levels can be reduced include one or more of the following: hsa-mir-135bNo1, hsa_mir_{—}95 right, hsa-mir-211-precNo2, hsa-mir-095-prec-4, hsa-mir-338No1, hsa-mir-26a-2No1, hsa-mir-199b-precNo2, hsa-mir-029c-prec, hsa-mir-106-prec-X, hsa-mir-425No1, hsa-mir-105-2No1, hsa-mir-030b-precNo1, hsa-mir-100No1, hsa-mir-026b-prec, hsa-mir-030a-precNo2, hsa-mir-020-prec, hsa-mir-328No1, hsa-mir-149-prec, hsa-miR-126*No1, hsa-mir-212-precNo1, hsa-mir-100-1/2-prec, hsa-mir-030c-prec, hsa-mir-030a-precNo1, hsa-mir-30c-1No1, hsa-mir-30c-2No1, hsa-mir-491No2, hsa-mir-099-prec-21, hsa-mir-494-No.1, hsa-mir-346-No.1, hsa_mir_{—}320_Hcd306 left., hsa-mir-320-No.2, hsa-mir-184-prec-No.2, hsa-mir-328-No.1, hsa-mir-130b-No.2, hsa-mir-196b-No.2 or combinations thereof:

Suitable compounds for inhibiting miR gene expression include double-stranded RNA (such as short- or small-interfering RNA or “siRNA”), antisense nucleic acids, enzymatic RNA molecules such as ribozymes, or molecules capable of forming a triple helix with the miR gene. Another class of inhibitor compound can cause hyper-methylation of the miR gene product promoter, resulting in reduced expression of the miR gene. Each of these compounds can be targeted to a given miR gene product to destroy, induce the destruction of, or otherwise reduce the level of the target miR gene product.

For example, expression of a given miR gene can be inhibited by inducing RNA interference of the miR gene with an isolated double-stranded RNA (“dsRNA) molecule which has at least 90%, for example at least 95%, at least 98%, at least 99% or 100-%, sequence homology with at least a portion of the miR gene product. In a particular embodiment, the dsRNA molecule is a “short” or small interfering RNA or “siRNA.”

siRNA useful in the present methods comprise short double-stranded RNA from about 17 nucleotides to about 29 nucleotides in length, or from about 19 to about 25 nucleotides in length. The siRNA comprise a sense RNA strand and a complementary antisense RNA strand annealed together by standard Watson-Crick base-pairing interactions (hereinafter “base-paired). The sense strand comprises a nucleic acid sequence which is substantially identical to a nucleic acid sequence contained within the target miR gene product. As used herein, a nucleic acid sequence in a siRNA which is “substantially identical” to a target sequence contained within the target mRNA is a nucleic acid sequence that is identical to the target sequence, or that differs from the target sequence by one or two nucleotides. The sense and antisense strands of the siRNA can comprise two complementary, single-stranded RNA molecules, or can comprise a single molecule in which two complementary portions are base-paired and are covalently linked by a single-stranded “hairpin” area.

The siRNA can also be engineered to contain certain “drug like” properties. Such modifications include chemical modifications for stability and cholesterol conjugation for delivery. Such modifications impart better pharmacological properties to the siRNA and using such modifications, pharmacologically active siRNAs can achieve broad biodistribution and efficient silencing of miRNAs in most tissues in vivo.

One or both strands of the siRNA can also comprise a 3′ overhang. As used herein, a “3′ overhang” refers to at least one unpaired nucleotide extending from the 3′-end of a duplexed RNA strand. Thus, in certain embodiments, the siRNA comprises at least one 3′ overhang of from 1 to about 6 nucleotides (which includes ribonucleotides or deoxyribonucleotides) in length, from 1 to about 5 nucleotides in length, from 1 to about 4 nucleotides in length, or from about 2 to about 4 nucleotides in length. In one embodiment, the 3′ overhang is present on both strands of the siRNA, and is 2 nucleotides in length. For example, each strand of the siRNA can comprise 3′ overhangs of dithymidylic acid (“TT”) or diuridylic acid (“uu”).

The siRNA can be produced chemically or biologically, or can be expressed from a recombinant plasmid or viral vector, as described above for the isolated miR gene products. Methods for synthesizing and validating a therapeutically effective siRNA engineered to silence miRNAs in vivo is described in Krützfeldt J, Rajewsky N, Braich R, Rajeev K G, Tuschl T, Manoharan M, Stoffel M. Silencing of microRNAs in vivo with ‘antagomirs’. Nature. 2005 Dec. 1; 438(7068):685-9.

Expression of a given miR gene can also be inhibited by an antisense nucleic acid. As used herein, an “antisense nucleic acid” refers to a nucleic acid molecule that binds to target RNA by means of RNA-RNA or RNA-DNA or RNA-peptide nucleic acid interactions, which alters the activity of the target RNA. Antisense nucleic acids suitable for use in the present methods are single-stranded nucleic acids (e.g., RNA, DNA, RNA-DNA chimeras, PNA) that generally comprise a nucleic acid sequence complementary to a contiguous nucleic acid sequence in a miR gene product. The antisense nucleic acid can comprise a nucleic acid sequence that is 50-100% complementary, 75-100% complementary, or 95-100% complementary to a contiguous nucleic acid sequence in a miR gene product. Without wishing to be bound by any theory, it is believed that the antisense nucleic acids activate RNase H or another cellular nuclease that digests the miR gene productlantisense nucleic acid duplex.

For example, in eukaryotes, RNA polymerase catalyzes the transcription of a structural gene to produce mRNA. A DNA molecule can be designed to contain an RNA polymerase template in which the RNA transcript has a sequence that is complementary to that of a preferred mRNA. The RNA transcript is termed an “antisense RNA”. Antisense RNA molecules can inhibit mRNA expression.

Antisense nucleic acids can also contain modifications to the nucleic acid backbone or to the sugar and base moieties (or their equivalent) to enhance target specificity, nuclease resistance, delivery or other properties related to efficacy of the molecule. Such modifications include cholesterol moieties, duplex intercalators, such as acridine, or one or more nuclease-resistant groups.

Antisense nucleic acids can be produced chemically or biologically, or can be expressed from a recombinant plasmid or viral vector, as described above for the isolated miR gene products. Exemplary methods for producing and testing are within the skill in the art.

Triple helix forming molecules can be used in reducing the level of a target miR gene activity. Nucleic acid molecules that can associate together in a triple-stranded conformation (triple helix) and that thereby can be used to inhibit translation of a target gene, should be single helices composed of deoxynucleotides. The base composition of these oligonucleotides must be designed to promote triple helix formation via Hoogsteen base pairing rules, which generally require sizeable stretches of either purines or pyrimidines on one strand of a duplex. Nucleotide sequences can be pyrimidine-based, which will result in TAT and CGC triplets across the three associated strands of the resulting triple helix. The pyrimidine-rich molecules provide bases complementary to a purine-rich region of a single strand of the duplex in a parallel orientation to that strand. In addition, nucleic acid molecules can be chosen that are purine-rich, for example, those that contain a stretch of G residues. These molecules will form a triple helix with a DNA duplex that is rich in GC pairs, in which the majority of the purine residues are located on a single strand of the targeted duplex, resulting in GGC triplets across the three strands in the triplex. Alternatively, the potential sequences that can be targeted for triple helix formation can be increased by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines on one strand of a duplex.

Liposomes may be used to deliver an miR gene product or miR gene expression-inhibiting compound (or nucleic acids comprising sequences encoding them) to a subject. Liposomes can also increase the blood half-life of the gene products or nucleic acids. Suitable liposomes for use in the invention can be formed from standard vesicle forming lipids, which generally include neutral or negatively charged phospholipids and a sterol, such as cholesterol. The selection of lipids is generally guided by consideration of factors, such as the desired liposome size and half-life of the liposomes in the blood stream. Varieties of methods are known for preparing liposomes and are well known to those skilled in the drug delivery arts.

In other embodiments, the pharmaceutical compositions of the invention comprise at least one miR expression inhibition compound. In a particular embodiment, the at least one miR gene expression inhibition compound is specific for a miR gene whose expression is greater in stem cells than control cells.

Pharmaceutical compositions of the present invention are characterized as being at least sterile and pyrogen-free. As used herein, pharmaceutical “compositions” or “formulations” include formulations for human and veterinary use. Methods for preparing pharmaceutical compositions of the invention are within the skill in the art, for example as described in Remington's Pharmaceutical Science, 17th ed., Mack Publishing Company, Easton, Pa. (1985), the entire disclosure of which is incorporated herein by reference.

Pharmaceutical compositions of the present invention are characterized as being at least sterile and pyrogen-free. The present pharmaceutical formulations comprise at least one miR gene product or miR gene expression inhibition compound (or at least one nucleic acid comprising sequences encoding them) (e.g., 0.1 to 90% by weight), or a physiologically acceptable salt thereof, mixed with a pharmaceutically-acceptable carrier. The pharmaceutical formulations of the invention can also comprise at least one miR gene product or miR gene expression inhibition compound (or at least one nucleic acid comprising sequences encoding them) which are encapsulated by liposomes and a pharmaceutically-acceptable carrier.

Pharmaceutical compositions of the invention can also comprise conventional pharmaceutical excipients and/or additives. Suitable pharmaceutical excipients include stabilizers, antioxidants, osmolality adjusting agents, buffers, and pH adjusting agents. Suitable additives include, e.g., physiologically biocompatible buffers (e.g., tromethamine hydrochloride), additions of chelants (such as, for example, DTPA or DTPA-bisamide) or calcium chelate complexes (such as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the invention can be packaged for use in liquid form, or can be lyophilized. For solid pharmaceutical compositions of the invention, conventional nontoxic solid pharmaceutically-acceptable carriers can be used; for example, pharmaceutical grades of mannitol, lactose, starch, magnesium stearate, sodium saccharin, talcum, cellulose, glucose, sucrose, magnesium carbonate, and the like.

The invention also contemplates a method for determining the efficacy of a therapeutic regimen inhibiting progression of disease in a subject. The method includes: (a) obtaining a first test sample from the subject's cells with an up-regulated miR gene product relative to control cells; (b) administering the therapeutic regimen to the subject; (c) obtaining a second test sample from the subject's tissue after a time period; and (d) comparing the levels of the up-regulated miR gene product in the first and the second test samples. A lower level of the up-regulated miR gene product in the second test sample as compared to the first test sample indicates that the therapeutic regimen is effective in the subject.

The invention also encompasses a method of identifying a stem cell specific anti-pathological agent. The method includes: (a) determining the expression level of at least one miR gene product which is over-expressed in a biological sample containing the subjects pathological tissue, thereby generating data for a pre-test expression level of said miR gene product; (b) contacting the biological sample with a test agent; (c) determining the expression level of the miR gene product in the biological sample after step (b), thereby generating data for a posttest expression level; and (d) comparing the post-test expression level to the pre-test expression level of said miR gene product. A decrease in the post-test expression level of the over-expressed miR gene product is indicative that the test agent has therapeutic properties.

In another embodiment, the method of identifying an stem cell-derived agent, includes: (a) determining the expression level of at least one miR gene product which is under-expressed in a biological sample containing stem cells, thereby generating data for a pre-test expression level of said miR gene product; (b) contacting the biological sample with a test agent; (c) determining the expression level of the miR gene product in the biological sample after step (b), thereby generating data for a post-test expression level; and (d) comparing the post-test expression level to the pre-test expression level of said miR gene product, wherein an increase in the post-test expression level of the under-expressed miR gene product is indicative that the test agent has therapeutic properties.

Suitable agents include, but are not limited to drugs (e.g., small molecules, peptides), and biological macromolecules (e.g., proteins, nucleic acids). The agent can be produced recombinantly, synthetically, or it may be isolated (i.e., purified) from a natural source. Various methods for providing such agents to a cell (e.g., transfection) are well known in the art, and several of such methods are described hereinabove. Methods for detecting the expression of at least one miR gene product (e.g., Northern blotting, in situ hybridization, RT-PCR, expression profiling) are also well known in the art. Several of these methods are also described hereinabove.

### EXEMPLIFICATION

The invention will now be illustrated by the following non-limiting examples.

### Example 1

### Identification of the Adult Stem Cell Marker Profile: Determination of the Stemness of the Adult Stem Cells

Immuno-histochemistry (IHC) and fluorescence-immunohistochemistry (FIHC) was used to profile a suspected adult stem cell source derived from the primordial mesenchymal bulb of the 3^{rd} molar region of both the upper and lower jaw. The IHC and FIHC of the prospected stem cells were contrasted with normal anatomical counterparts from this same region that were differentiated. The profile was based on the consensus profile of human embryonic stem cells (FIG. 4 and from the group Oct-3/4, SSEA1, SSEA3, SSEA4, Tra-1-60, TRA-1-81, SOX2, NANOG, CD44, CD34, CD9, CD133, CD117, CD4, CD8, MART, and CD24).

### Materials and Methods.

Tissue Procurement—The stem cell samples analyzed were obtained from seven peripubertal humans, three females and four males, ranging in ages from 13-18 years old. These patients were undergoing voluntary prophylactic removal of this tissue and this tissue was obtained with consent. Normal anatomical counterpart tissues were obtained from eight postpubertal adults ranging age from 24-38 years old. These tissues were obtained by consent from patients undergoing prophylactic removal of this tissue. In all cases samples were placed in ice cold tissue support media upon removal form the patient. All tissues were processed within four hours of being obtained. All tissue were anonimized and untraceable to the donor source. All HIPAA and CITI guide lines, and all applicable laws were strictly adhered to in the obtaining of tissues.

Tissue Processing—Portions of the tissue were 1] frozen in liquid nitrogen, 2] excised and dissipated for culturing, and 3] fixed and paraffin embedded. Tissue that was embedded or frozen in liquid nitrogen were further processed for evaluation by either standard structural microscopic analysis, e.g. H and E staining, or processed for IHC or FIHC. The procedures and markers are as outlined in Carpenter M K, Rosier E, Rao M S. Characterization and differentiation of human embryonic stem cells: Cloning Stem Cells. 2003; 5(1):79-88; Baal N, Reisinger K, Jahr H, Bohle R M, Preissner K T, Zygmunt M T. Expression of transcription factor Oct-4 and other embryonic genes in CD133 positive cells from human umbilical cord blood. Thromb Haemost. 2004 October; 92(4): 767-75; Zou G M, Chen J J, Yoder M C, Wu W, Rowley J D. Knockdown of Pu.1 by small interfering RNA in CD34+ embryoid body cells derived from mouse ES cells turns cell fate determination to pro-B cells. Proc Natl Acad Sci USA. 2005 Sep. 13; 102(37):13236-41; Zhao Y, Wang H, Mazzone T. Identification of stem cells from human umbilical cord blood with embryonic and hematopoietic characteristics. Exp Cell Res. 2006 Aug. 1; 312(13):2454-64; Miki T, Mitamura K, Ross M A, Stolz D B, Strom S C. Identification of stem cell marker-positive cells by immunofluorescence in term human amnion. J Reprod Immunol. 2007 October; 75(2):91-6; http://www.millipore.com/catalogue/item/scr018; http://www.millipore.com/catalogue/item/scr002. Hematoxylin/Eosin staining is well known to those skilled in the arts of histology. Cell culturing was accomplished via the use of a serum-free media. Relative levels of the markers were determined via counting under a microscope or via quantitative fluorescence.

### Results and Discussion

Contrasting Expression: The results of the expression of embryonic stem cell markers in the stem cells derived from the patient sources are outlined in Table 1, below. Table 1 indicates that the embryonic stem cell markers, those that by consensus define embryonic stem cells, are strongly expressed in the target adult stem cells from the primordial mesenchymal stem cell bulb of the pre-third molar. Additionally, when one contrasts these results with hematopoietic stem cells, the profile is remarkably different and indeed, this profiling specifically excludes CD34+ stem cells of the hematopoietic origin. Additionally, this profiling indicates that these stem cells have not attained any notable level of differentiation when contrasted with the differentiated counterpart. Lastly, when using these markers as a definition of relative stemness in contrast with embryonic stem cells, on a scale of 0-10, 10 being totipotential or neozygote, these mesenchymal stem cells appear to be more pluripotent than hematopoietic stem cells as indicated in the bottom row of the table below.

These data outline a compilation of stem cell markers evaluated by our research efforts to contrast the oral mesenchymal stem cells with known markers of “stemness” as defined by the current literature in stem cell research on embryonic stem cells. The markers were chosen on the basis of providing information on the expression of those markers that are considered highly significant by their expression, highly significant by their absence and those that represent hematopoietic sources of stem cells. References for these markers are listed in the table bottom row ((Slavin S, Kurkalli B G, Karussis D. The potential use of adult stem cells for the treatment of multiple sclerosis and other neurodegenerative disorders. Clin Neurol Neurosurg. 2008 Mar. 5; Epub PMID: 18325660; Sahin M B, Schwartz R E, Buckley S M, Heremans Y, Chase L, Hu W S, Verfaillie C M. Isolation and characterization of a novel population of progenitor cells from unmanipulated rat liver. Liver Transpl. 2008 March; 14(3):333-45; King C C, Beattie G M, Lopez A D, Hayek A. Generation of definitive endoderm from human embryonic stem cells cultured in feeder layer-free conditions. Regen Med. 2008 March; 3(2):175-80; Fransioli J, Bailey B, Gude N A, Cottage C T, Muraski J A, Emmanuel G, Wu W, Alvarez R, Rubio M, Ottolenghi S, Schaefer E, Sussman M A. Evolution of The c-kit Positive Cell Response to Pathological Challenge in the Myocardium. Stem Cells. 2008 Feb. 28; Epub PMID: 18308948; Park Y B, Kim Y Y, Oh S K, Chung S G, Ku S Y, Kim S H, Choi Y M, Moon S Y. Alterations of proliferative and differentiation potentials of human embryonic stem cells during long-term culture. Exp Mol Med. 2008 Feb. 29; 40(1):98-108; Agarwal S, Holton K L, Lanza R. Efficient Differentiation of Functional Hepatocytes from Human Embryonic Stem Cells. Stem Cells. 2008 Feb. 21; Epub PMID; Garber K. Epithelial-to-mesenchymal transition is important to metastasis, but questions remain. J Natl Cancer Inst. 2008 Feb. 20; 100(4):232-3, 239; Toyooka Y, Shimosato D, Murakami K, Takahashi K, Niwa H. Identification and characterization of subpopulations in undifferentiated ES cell culture. Development. 2008 March; 135(5):909-18; Kerr C L, Hill C M, Blumenthal P D, Gearhart J D. Expression of pluripotent stem cell markers in the human fetal ovary. Hum Reprod. 2008 March; 23(3):589-99; Tsuneyoshi N, Sumi T, Onda H, Nojima H, Nakatsuji N, Suemori H. PRDM14 suppresses expression of differentiation marker genes in human embryonic stem cells. Biochem Biophys Res Commun. 2008 Mar. 21; 367(4):899-905; Kolodziejska K M, Ashraf H N, Nagy A, Bacon A, Frampton J, Xin H B, Kotlikoff M I, Husain M. c-Myb Dependent Smooth Muscle Cell Differentiation. Circ Res. 2008 Jan. 10; Epub PMID: 18187733; Dhara S K, Hasneen K, Machacek D W, Boyd N L, Rao R R, Stice S L. Human neural progenitor cells derived from embryonic stem cells in feeder-free cultures. Differentiation. 2008 Jan. 3; [Epub PMID: 18177420; Cholette J M, Blumberg N, Phipps R P, McDermott M P, Gettings K F, Lerner N B. Developmental changes in soluble CD40 ligand. J Pediatr. 2008 January; 152(1):50-4, 54.e1; Nadri S, Soleimani M, Kiani J, Atashi A, Izadpanah R. Multipotent mesenchymal stem cells from adult human eye conjunctiva stromal cells. Differentiation. 2008 March; 76(3):223-31; Barker N, Clevers H. Tracking down the stem cells of the intestine: strategies to identify adult stem cells. Gastroenterology. 2007 December; 133(6): 1755-60; Lakshmipathy U, Hart R P. Concise review: MicroRNA expression in multipotent mesenchymal stromal cells. Stem Cells. 2008 February; 26(2):356-63; Kerr C L, Hill C M, Blumenthal P D, Gearhart J D. Expression of pluripotent stem cell markers in the human fetal testis. Stem Cells. 2008 February; 26(2):412-21; Babaie Y, Herwig R, Greber B, Brink T C, Wruck W, Groth D, Lehrach H, Burdon T, Adjaye J. Analysis of Oct4-dependent transcriptional networks regulating self-renewal and pluripotency in human embryonic stem cells. Stem Cells. 2007 February; 25(2):500-10; Ai C, Todorov I, Slovak M L, Digiusto D, Forman S J, Shih C C. Human marrow-derived mesodermal progenitor cells generate insulin-secreting islet-like clusters in vivo. Stem Cells Dev. 2007 October; 16(5): 757-70; Yu J, Vodyanik M A, Smuga-Otto K, Antosiewicz-Bourget J, Frane J L, Tian S, Nie J, Jonsdottir G A, Ruotti V, Stewart R, Slukvin I I, Thomson J A. Induced pluripotent stem cell lines derived from human somatic cells. Science. 2007 Dec. 21; 318(5858):1917-20; Abzhanov A, Rodda S J, McMahon A P, Tabin C J. Regulation of skeletogenic differentiation in cranial dermal bone. Development. 2007 September; 134(17):3133-44).

TABLE 1
ADULT STEM CELL MARKERS - EXPRESSION OF TRANSCRIPTION
FACTORS, AND EMBRYONIC ANTIGEN MARKERS OF ADULT STEM
CELLS VERSUS EMBRYONIC STEM CELLS AND FULLY DIFFERENTIATED
AND ANATOMICALLY CORRECT DIFFERENTIATED TISSUE

| | FULLY | | | |
|---|---|---|---|---|
| | DIFFERENTIATED | ADULT STEM | | |
| | ANATOMICALLY | ORAL | HUMAN | |
| | CORRECT | MESENCHYMAL | EMBRYONIC | HEMATOPOIETIC |
| | COUNTERPART\hat{ } | STEM CELL\hat{ } | STEM CELL | STEM CELLS |
| OCT-3/4 | − | +++ | ++++ | +/− |
| CD9 | − | ++ | +++ | ++ |
| CD133 | − | +++ | +++ | ND |
| CD44 | − | − | − | +++ |
| SOX2 | − | +++ | +++ | − |
| SSEA3/4 | − | +++ | ++++ | − |
| NANOG | − | +++ | +++ | − |
| CD34 | − | − | − | ++++ |
| CD117 | − | − | − | +++ |
| CD4 | − | − | − | − |
| CD8 | − | − | − | − |
| CD24 | ND | ND | − | − |
| Relative degree of | 0.5 | 8.7 | 9.9 | 7.2 |
| “stemness” (1-10 | | | | |
| where 10 = human | | | | |
| embryonic stem cell | | | | |
| or near totipotency) | | | | |

\hat{ }Data based on our work for this application; ND = Not done; + = relative increased detection of this marker; − = none detected or negative.

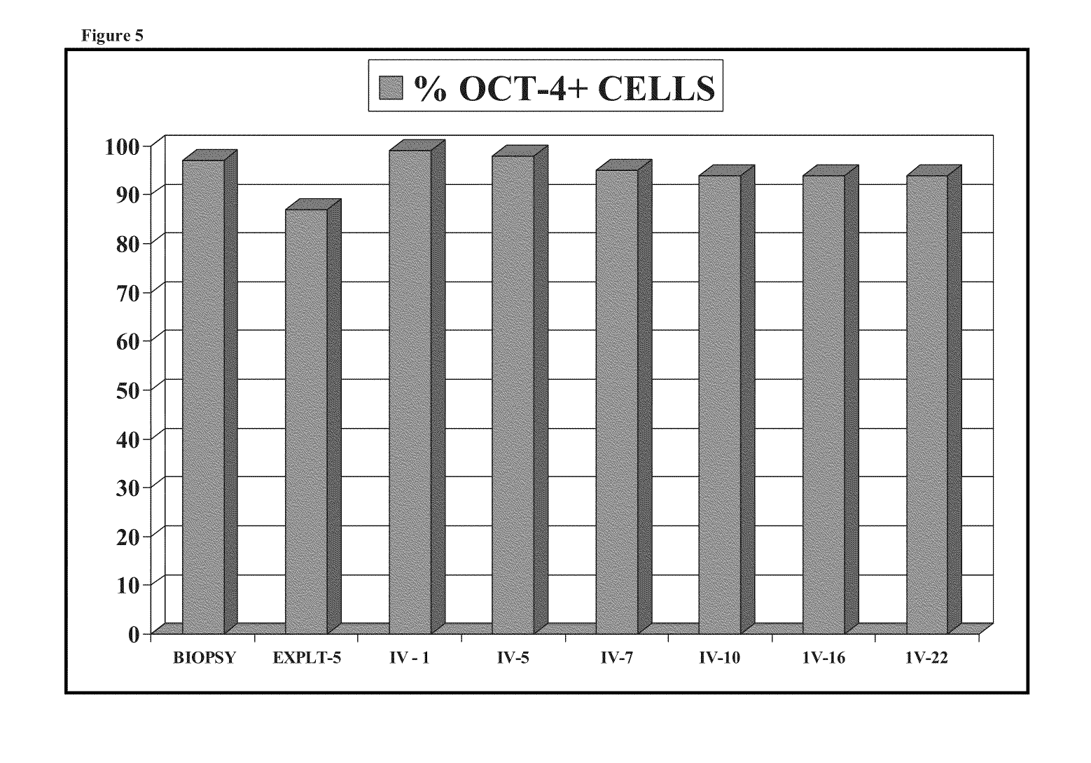

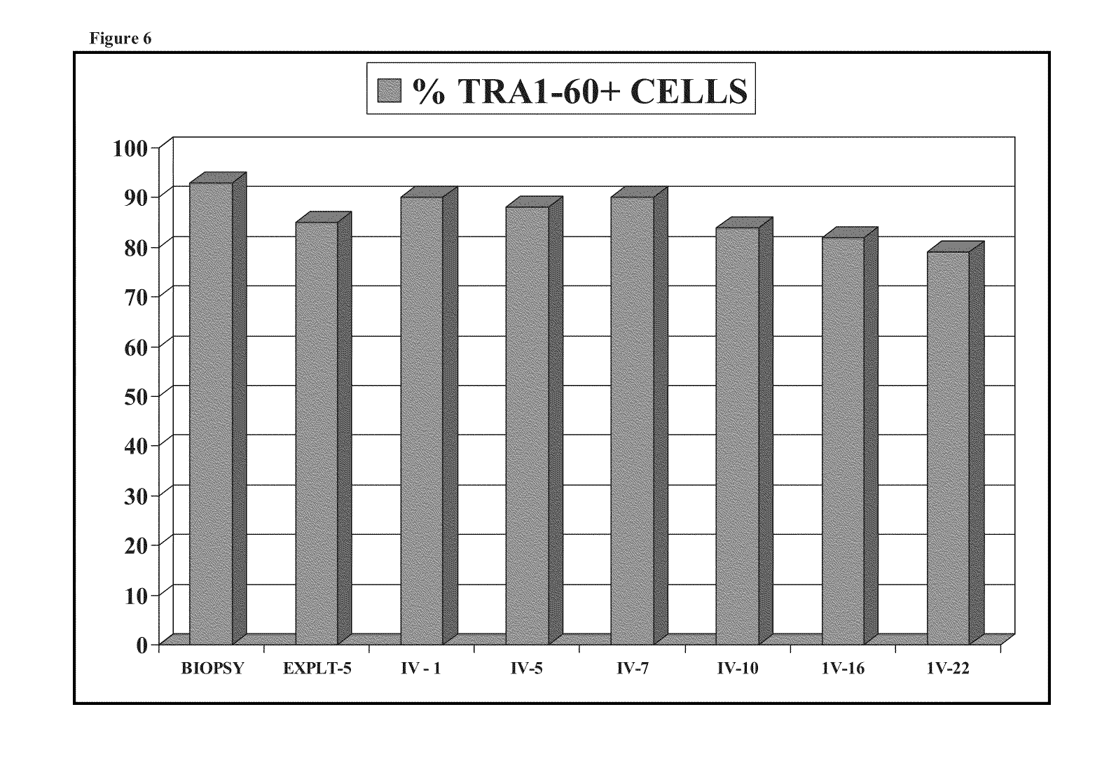

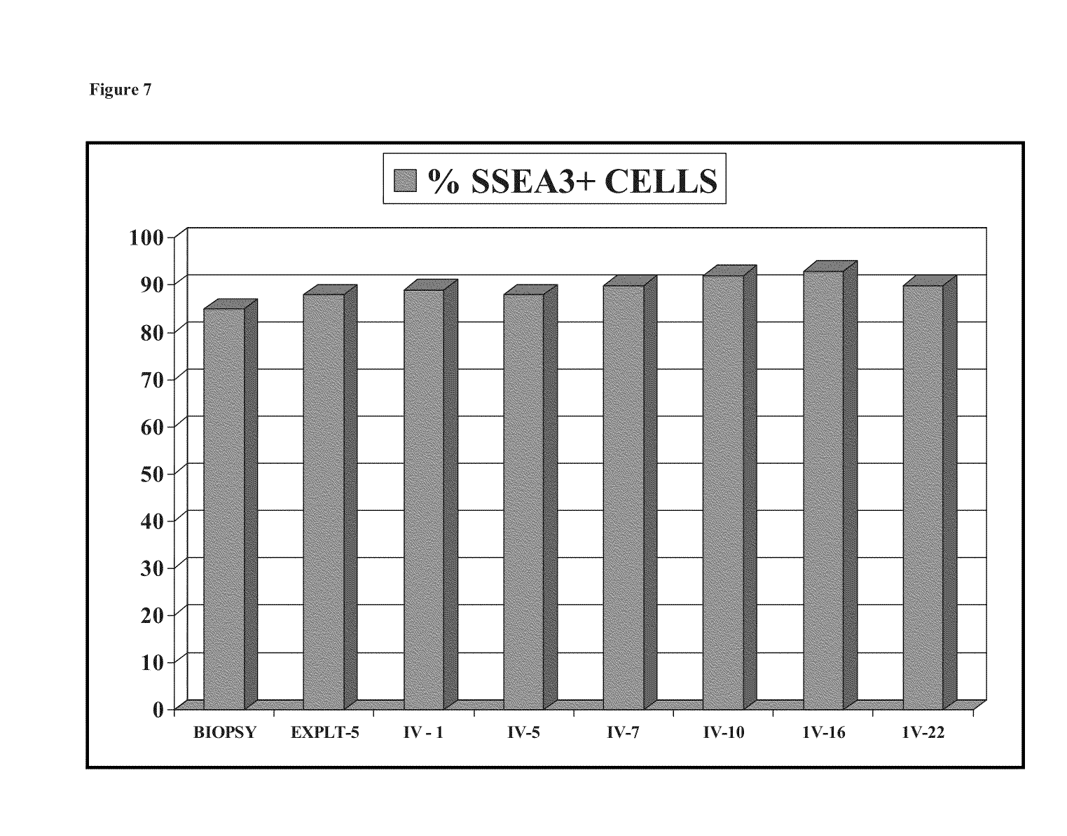

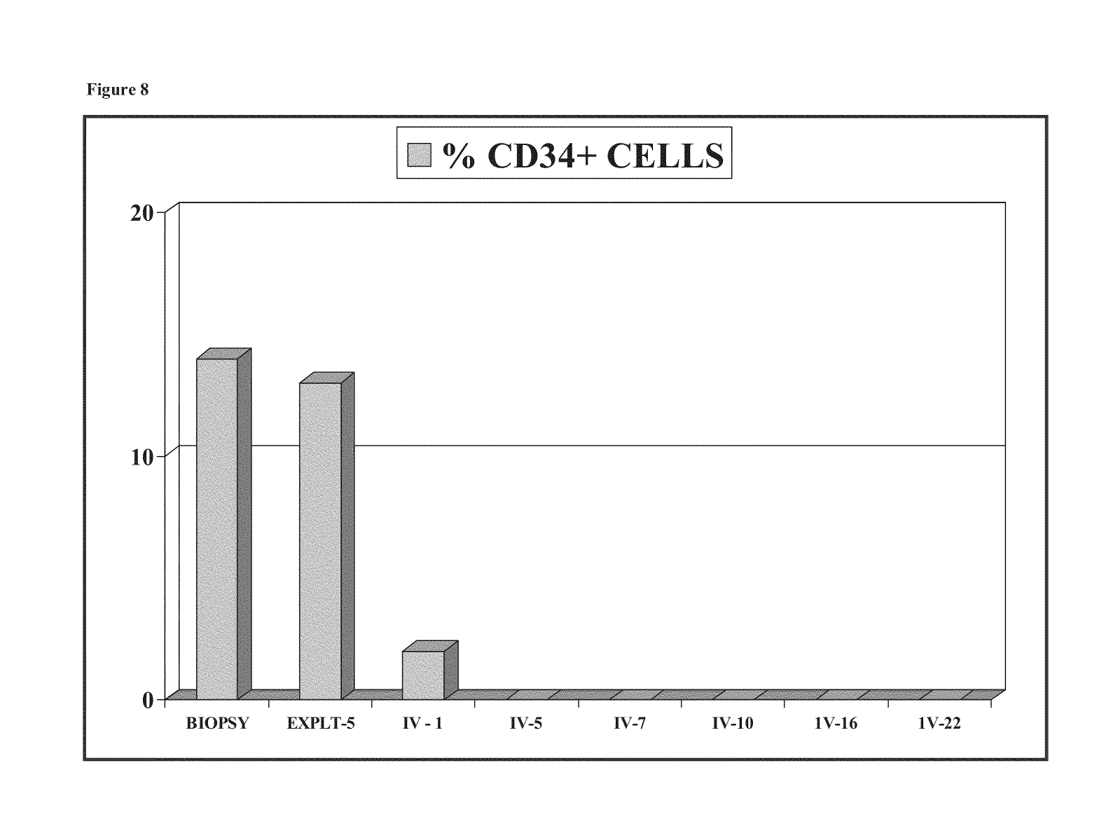

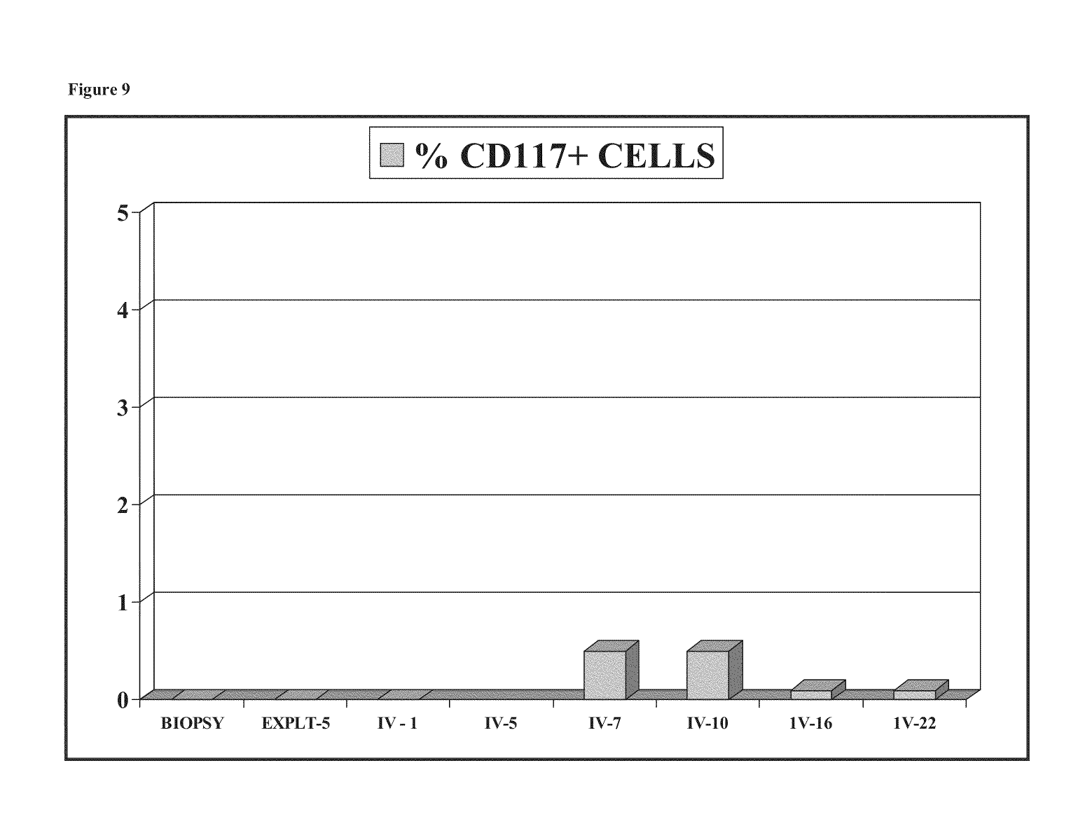

Marker Retention: In addition to the evaluation the initial biopsy material, dissipated cells were placed in cell culture medium and incubated for a period of 22 days at 37 C. Cell culturing was accomplished via the use of a serum-free, xenobiotic material-free, organotypic matrix-free media that remains the trade secret of Clue Genomics. The results of selected key markers are shown in FIGS. 5-9. These figures indicate that key markers of stemness can be retained over this critical period when many cells stochastically differentiate (FIGS. 5-7, Oct-3/4, TRA-1-60, and SSEA-3, respectively). Moreover, these cells do not acquire phenotypic markers that define their path to hematopoietic-like cells or other differentiated states (FIGS. 8 and 9, CD34 and CD117, respectively).

### Example 2

### Identification of the microRNA Signature that Discriminates Adult Stem Cells from Their Anatomically Correct Differentiated Counterpart

Microarray analysis is a relatively new and powerful tool to discern expression profiles, and levels of expression of multiple, in this case, 573 known miRs simultaneously along with the proper controls tissues. This provides high reliability and diminishes noise levels between assays as well as provides for an extremely low standard deviation between samples, within treatments, and between different time points. The microarray chips utilized in the analysis represent the largest single chip array available in the world today. This chip is a custom production available to few persons at this time and thus, the results of our analysis provide the single greatest comprehension of stem cell signature composites in the world. The end reliability of this signature derived from this analysis is thus extremely high and provides for potential remarkable utility of the signature in defining stem cells, identifying stem cells, modifying stem cells toward desired differentiated end points, component miR elixirs that may be used to treat diseases in vivo, and to diagnose specific pathologies and their probability of repair.

### Materials and Methods

Tissue Procurement—The stem cell samples analyzed were obtained from seven peripubertal humans, three females and four males, ranging in ages from 13-18 years old. These patients were undergoing voluntary prophylactic removal of this tissue and this tissue was obtained with consent. Normal anatomical counterpart tissues were obtained from eight postpubertal adults ranging age from 24-38 years old. These tissues were obtained by consent from patients undergoing prophylactic removal of this tissue. In all cases samples were placed in ice cold tissue support media upon removal form the patient. All tissues were processed within four hours of being obtained. All tissue were anonimized and untraceable to the donor source. All HIPAA and CITI guide lines and all applicable laws were strictly adhered to in the obtaining of tissues.

Tissue Processing—Portions of the tissue were 1] frozen in liquid nitrogen, 2] excised and dissipated for culturing, and 3] fixed and paraffin embedded. Tissue that was embedded or frozen in liquid nitrogen were further processed for RNA extraction. The complete method of RNA preparation is outlined previously and this methodology is provided by complete reference in Calin G A, Croce C M. MicroRNA signatures in human cancers. Nat Rev Cancer. 2006 November; 6(11):857-66; P, Krek A, Zavolan M, Macino G, Rajewsky N. Cell-type-specific signatures of microRNAs on target mRNA expression. Proc Natl Acad Sci USA. 2006 Feb. 21; 103(8):2746-51; Yin J Q, Zhao R C, Morris K V. Profiling microRNA expression with microarrays. Trends Biotechnol. 2008 February; 26(2): 70-6; Liu C G, Calin G A, Meloon B, Gamliel N, Sevignani C, Ferracin M, Dumitru C D, Shimizu M, Zupo S, Dono M, Alder H, Bullrich F, Negrini M, Croce C M. An oligonucleotide microchip for genome-wide microRNA profiling in human and mouse tissues. Proc Natl Acad Sci USA. 2004 Jun. 29; 101 (26):9740-4; Volinia S, Calin G A, Liu C G, Ambs S, Cimmino A, Petrocca F, Visone R, Iorio M, Roldo C, Ferracin M, Prueitt R L, Yanaihara N, Lanza G, Scarpa A, Vecchione A, Negrini M, Harris C C, Croce C M. A microRNA expression signature of human solid tumors defines cancer gene targets. Proc Natl Acad Sci USA. 2006 Feb. 14; 103(7): 2257-61.

Microarray analysis—Microarray analysis was performed as previously described (Liu, C. G., et al., Proc. Natl. Acad. Sci. U.S.A. 101:9740-9744, 2004). Briefly, 5 pgm of total RNA was hybridized with miRNA microarray chips containing 573 miR probes in triplicate. Specifically, these chips contain gene-specific 40-mer oligonucleotide probes, spotted by contacting technologies and covalently attached to a polymeric matrix, which were generated from 573 human miRNAs. The microarrays were hybridized in 6×SSPE (0.9 M NaCl/60 mM NaH2P04-H₂O/8 mM EDTA, pH 7.4)/30% formarnide at 25 C for 18 hr, washed in 0.75×TNT (Tris-HCl/NaCl/Tween 20) at 37 C for 40 min, and processed using a method of direct detection of biotin-containing transcripts by streptayidin-Alexa647 conjugate (Molecular Probes, Carlsbad, Calif.). Processed slides were scanned using a PerkinElmer ScanArray XL5K Scanner, with the laser set to 635 nm, at Power 80 and PMT 70 setting, and a scan resolution of 10 μm. An average value of the three spot 10 replicates for each miRNA was normalized and analyzed in BRB-AnayTools version 3.2.3. After excluding negative values with hybridization intensity below background normalization was performed on each chip using median normalization methodology and normalization to median array as reference. Genes that were differently expressed among groups were identified using t- or F-test and genes were considered statistically significant if their p-value was less than 0.001. A global test of whether the expression profiles differed between the groups was also performed by per mutating the labels of which arrays corresponded to which groups. For each permutation, the p-values were re-computed and the number of genes significant at the p<0.001 level was noted. The proportion of the permutations that gave at least as many significant genes as with the actual data was the significance level of the global test.

### Results and Discussion

Table 2a and 2b, below, represent the 75 most significantly altered microRNAs in the adult oral mesenchymal stem cells in contrast to the anatomically correct differentiated counterpart or differentiated tissue extracted from the same anatomical site. The stem cell analysis represents six donor patients (3 male and 3 female, ages 12-18 years old) while the anatomically identical fully differentiated counterparts are represented by 8 donors (4 male and 4 female between, ages of 22-36 years old). These microRNAs are listed in order of significance (by p-value, e.g. has-mir-135bNo1 has a p-value of <1.5771×10⁻¹⁰) and level of expression (Table section 2a) making the over expression of this microRNA in this tissue highly significant in contrast to its anatomically correct counterpart or under-expression or not expressed (Table section 3b, by p-value, e.g. has-mir-517b has a p-value of <1.04361×10⁻¹³) making this microRNA effectively not expressed in the stem cell population in contrast to its normal anatomical counterpart. The interface of the JAVA program we implemented takes a list of gap files as input, and gives an output dataset file. We used the “gpr” table to identify the versions of miRNA chips and then compile the list of miRNAs whose expression needs to be extracted from the database. The output dataset is a matrix of float values and has the columns' headers formed by the gpr of the list, and the rows' headers by the reporter name of the miRNAs. Once the miRNA list is created, we store the list in memory in a structure that hashes the Reporter name. Then we focus on each single gpr. For each gpr in the list we locate in memory the miRNAs contained in the table. The algorithm currently reports the expression values in different ways: the average of the miRNA replicates on each chip or the geometric mean of the miRNAs replicates. This information is in turn available for the replicate values that corresponds the F635Median field in the Spot table or for the F635Median-B635 field. Usually the dataset contains the values described after a Log 2 operation. This is useful to obtain values in a smaller range. The Average and the Geometric mean we calculate are only on zero flagged values. That means there are no survey errors on the miRNAs replicate. The application tracks also the errors detected for the miRNA replicates. The number of replicate with flag −50 and −100 are thus stored in memory for successive adjustments. Proceeding as described, we fill the output dataset matrix column by column, and the final dimension of the matrix is [number of miRNAs]×[number of .gpr in the list]. The error values are treated differently depending by the type of flag and the ratio: (number of 0 flag/number of error flag). We defined “not available” values when a miRNA has more replicates with a −100 flag than half the total number of its replicates. Considering a row in the output matrix, if there were not available values, we substituted that value with the mean of the other values of that row. The influence of the −50 flags is different and depends on which information the system calculates. Table 2a indicates the elevation of the stem cell miR expression over the normal cell expression levels. The relative fractional expression of the stem cells is indicated in the column 3. Fold change of the normal to stem cell is relatively fractional as indicated by the values being less than 1 in this column. It is noteworthy that all of the p-values for elevated expression of the stem cell miRs are <1.5×10⁻⁵.

TABLE 2
SELECT ADULT STEM CELL MIR EVALUATION BY MICROARRAY
Table 2a - 40 MOST SIGNIFICANTLY ELEVATED MICRORNAs
COMPARED TO ANATOMICALLY CONTROL

| | | Fold change NORMAL | |
|---|---|---|---|
| MIR NUMBER | p-value | VS STEM CELL | ASCENTION NUMBER\hat{ } |
| hsa-mir-135bNo1 | 1.5771E−10 | 0.558103012 | MIMAT0000758 |
| hsa-mir-95 right | 1.9938E−10 | 0.612285109 | MIMAT0000094 |
| hsa-mir-211-precNo2 | 5.6905E−08 | 0.587943666 | MI0000287 |
| hsa-mir-095-prec-4 | 8.96927E−08 | 0.596870279 | MI0000097 |
| hsa-mir-338No1 | 1.40435E−07 | 0.274546165 | MIMAT0004701 |
| hsa-mir-26a-2No1 | 2.34135E−07 | 0.226108009 | MIMAT0000082 |
| hsa-mir-199b-precNo2 | 3.53938E−07 | 0.154664048 | MI0000282 |
| hsa-mir-029c-prec | 4.50624E−07 | 0.361748421 | MI0000735 |
| hsa-mir-106-prec-X | 5.98549E−07 | 0.277199956 | MI0000734 |
| hsa-mir-425No1 | 6.36884E−07 | 0.290533708 | MIMAT0003393 |
| hsa-mir-105-2No1 | 6.52432E−07 | 0.26251363 | MIMAT0000102 |
| hsa-mir-369No1 | 7.15218E−07 | 0.413476221 | MIMAT0001621 |
| hsa-mir-132-precNo2 | 7.20309E−07 | 0.76063751 | MI0000449 |
| hsa-mir-181b-2No2 | 8.42685E−07 | 0.826342757 | MIMAT0000257 |
| hsa-mir-030b-precNo1 | 1.13445E−06 | 0.134943683 | MI0000441 |
| hsa-mir-100No1 | 1.23992E−06 | 0.135664375 | MIMAT0000098 |
| hsa-mir-026b-prec | 1.46949E−06 | 0.15539514 | MI000084 |
| hsa-mir-030a-precNo2 | 1.86141E−06 | 0.526512938 | MI000088 |
| hsa-mir-020-prec | 2.08919E−06 | 0.276480695 | MI000076/1519 |
| hsa-mir-328No1 | 3.09784E−06 | 0.235649892 | MIMAT0000752 |
| hsa-mir-192-2/3No1 | 3.51925E−06 | 0.407748715 | MIMAT0000222 |
| hsa-mir-149-prec | 3.68908E−06 | 0.387956478 | MI0000478 |
| hsa-mir-30eNo2 | 3.89173E−06 | 0.564334205 | MI MAT0000692 |
| hsa-miR-126*No1 | 4.55261E−06 | 0.308707777 | MI0000471 |
| hsa-mir-212-precNo1 | 4.98105E−06 | 0.188475527 | MI0000288 |
| hsa-mir-337No1 | 5.44853E−06 | 0.534699488 | MIMAT0004695 |
| hsa-mir-125b-2-precNo2 | 6.5741E−06 | 0.359398288 | MI0000470 |
| hsa-mir-100-1/2-prec | 7.43672E−06 | 0.191004203 | MI0000692 |
| hsa-mir-030c-prec | 9.71313E−06 | 0.19420716 | MI0000736/254 |
| hsa-mir-126No2 | 9.72401E−06 | 0.330701547 | MI0000444 |
| hsa-mir-410No1 | 9.74202E−06 | 0.592086663 | MIMAT0002171 |
| hsa-mir-450-1 | 9.99266E−06 | 0.906966477 | MIMAT0001545 |
| hsa-mir-030a-precNo1 | 1.06111E−05 | 0.202736558 | MI0000088 |
| hsa-mir-184-precNo2 | 1.16041E−05 | 0.309530475 | MI0000481 |
| hsa-mir-030d-precNo2 | 1.20192E−05 | 0.252396618 | MI0000255 |
| hsa-mir-133bNo2 | 1.25098E−05 | 0.296981702 | MIMAT0000770 |
| hsa-mir-30c-1No1 | 1.27356E−05 | 0.180648065 | MIMAT0000244/MI0000736 |
| hsa-mir-361No1 | 1.39061E−05 | 0.248089985 | MIMAT0000703 |
| hsa-mir-30c-2No1 | 1.42283E−05 | 0.169512609 | MIMAT0000244/MI0000254 |

\hat{ }Based on Sanger MicroRNA Data Base and Global Terminology and Sequence Standardization http://microrna.sanger.ac.uk

### Example 3

### Identification of the microRNA Signature that Discriminates Adult Stem Cells from the Normal Tissues of Trachea, Kidney, Liver, Colon, Small Intestine, Pancreas, Stomach, Esophagus, Bladder, Prostate, Thyroid, Heart, Skeletal Muscle, Testes, Cervix, Ovary, Uterus, Breast, Thymus, Lung, Spleen, Adipose tissue, Lymph Node, Brain, Adrenal, and Placenta

We applied microarray analysis to discern expression profiles, and levels of expression of multiple, in this case, 573 known human miRs, simultaneously, along with the proper controls tissues, to a contrast with 27 different differentiated tissues. The microarray chips utilized in the analysis represent the largest single chip array available in the world today. Interchip controls were used to provide for between chip variances. These chips are custom productions available to few persons at this time and thus, the results of our analysis provide the single greatest comprehension of stem cell signature composites in the world. The end reliability of this signature derived from this analysis is thus extremely high. The differential expression of the differentiated tissues vs the stem cells provides us with insights toward the proper relative signatures that may be utilized in differentiating the stem cells in vitro in order to obtain clinically valuable terminally differentiated tissues that may be applied therapeutically to pathological diagnoses. For example, the differential signature of the stem cells in contrast to the normal anatomically correct control tissue may specifically yield miR profiles that provide for enhanced stem cell expression in vivo when delivered intravenously. Thus contrasting signatures for all tissues provides for potential remarkable utility of the signature in defining stem cells, identifying stem cells, modifying stem cells toward desired differentiated end points, component miR elixirs that may be used to treat diseases in vivo, and to diagnose specific pathologies and their probability of repair.

### Materials and Methods

Tissue Procurement—The stem cell samples analyzed were obtained from seven peripubertal humans, three females and four males, ranging in ages from 13-18 years old. These patients were undergoing voluntary prophylactic removal of this tissue and this tissue was obtained with consent. Normal anatomical counterpart tissues were obtained from eight postpubertal adults ranging age from 24-38 years old. These tissues were obtained by consent from patients undergoing prophylactic removal of this tissue. In all cases, samples were placed in ice cold tissue support, media upon removal from the patient. All tissues were processed within four hours of being obtained. Tissues from the 26 different differentiated tissues were obtained through an IRB approved tissue procurement protocol. All tissue were anonimized and untraceable to the donor source. All HIPAA and CITI guidelines and all applicable laws were strictly adhered to in the obtaining of tissues.

Tissue Processing—Portions of the tissue were 1] frozen in liquid nitrogen, 2] excised and dissipated for culturing, and 3] fixed and paraffin embedded. Tissue that was embedded or frozen in liquid nitrogen were further processed for RNA extraction. The complete method of RNA preparation is provided by reference and is outlined previously in Calin G A, Croce C M. MicroRNA signatures in human cancers. Nat Rev Cancer. 2006 November; 6(11):857-66; P, Krek A, Zavolan M, Macino G, Rajewsky N. Cell-type-specific signatures of microRNAs on target mRNA expression. Proc Natl Acad Sci USA. 2006 Feb. 21; 103(8):2746-51; Yin J Q, Zhao R C, Morris K V. Profiling microRNA expression with microarrays. Trends Biotechnol. 2008 February; 26(2):70-6; Liu C G, Calin G A, Meloon B, Gamliel N, Sevignani C, Ferracin M, Dumitru C D, Shimizu M, Zupo S, Dono M, Alder H, Bullrich F, Negrini M, Croce C M. An oligonucleotide microchip for genome-wide microRNA profiling in human and mouse tissues. Proc Natl Acad Sci USA. 2004 Jun. 29; 101(26):9740-4; Volinia S, Calin G A, Liu C G, Ambs S, Cimmino A, Petrocca F, Visone R, Iorio M, Roldo C, Ferracin M, Prueitt R L, Yanaihara N, Lanza G, Scarpa A, Vecchione A, Negrini M, Harris C C, Croce C M. A microRNA expression signature of human solid tumors defines cancer gene targets. Proc Natl Acad Sci USA. 2006 Feb. 14; 103(7):2257-61.

Microarray analysis—Microarray analysis was performed as previously described (Liu, C. G., et al., 30 Proc. Natl. Acad. Sci. U.S.A. 101:9740-9744, 2004). Briefly, 5 pg of total RNA was hybridized with miRNA microarray chips containing 573 miR probes in triplicate. Specifically, these chips contain gene-specific 40-mer oligonucleotide probes, spotted by contacting technologies and covalently attached to a polymeric matrix, which were generated from 573 human miRNAs. The microarrays were hybridized in 6×SSPE (0.9 M NaCl/60 mM NaH2P04-H₂O/8 mM EDTA, pH 7.4)/30% formamide at 25 C for 18 hr, washed in 0.75×TNT (Tris-HCl/NaCl/Tween 20) at 37 C for 40 min, and processed using a method of direct detection of biotin-containing transcripts by streptavidin-Alexa647 conjugate (Molecular Probes, Carlsbad, Calif.). Processed slides were scanned using a PerkinElmer ScanArray XL5K Scanner, with the laser set to 635 nm, at Power 80 and PMT 70 setting, and a scan resolution of 10 μm. An average value of the three spot 10 replicates for each miRNA was normalized and analyzed in BRB-AnayTools version 3.2.3. After excluding negative values with hybridization intensity below background normalization was performed on each chip using median normalization methodology and normalization to median array as reference. Genes that were differently expressed among groups were identified using t- or F-test and genes were considered statistically significant if their p-value was less than 0.001. A global test of whether the expression profiles differed between the groups was also performed by permutating the labels of which arrays corresponded to which groups. For each permutation, the p values were re-computed and the number of genes significant at the <0.001 level was noted. The proportion of the permutations that gave at least as many significant genes as with the actual data was the significance level of the global test. Also see table description legend Tables 3a and 3b.

### Results and Discussion

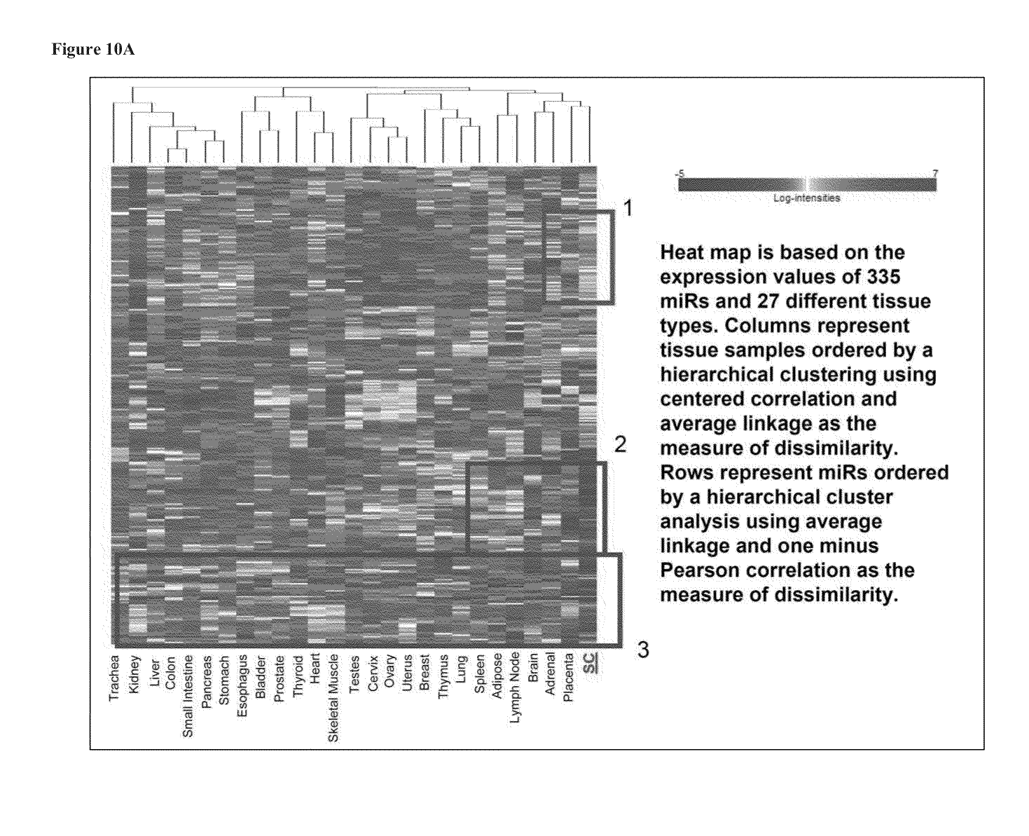

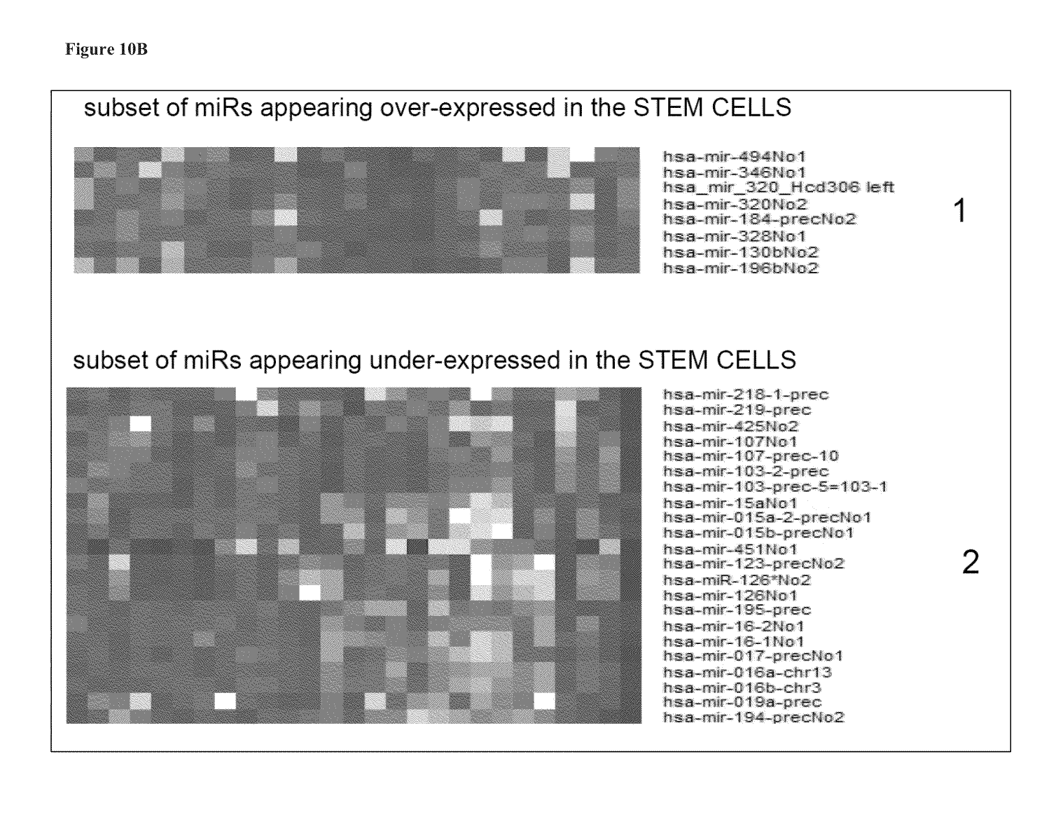

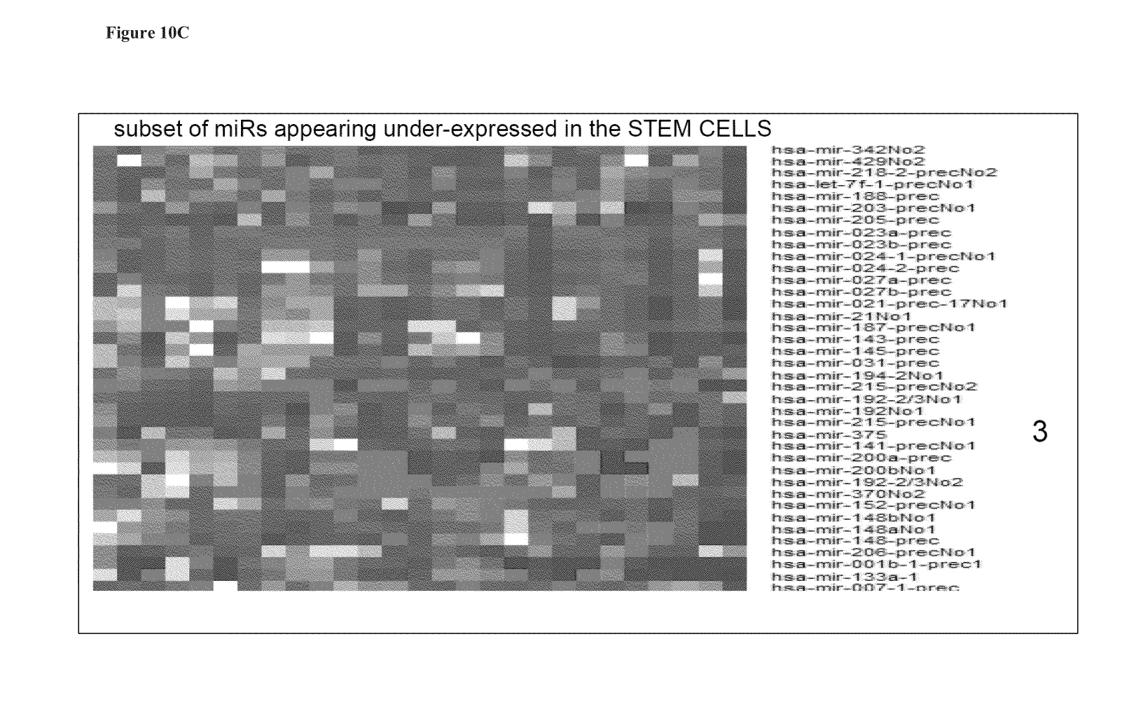

FIG. 10 indicates the results of the relative expression of 573 miRs for all 27 tissues vs the stem cell (SC). This image FIG. 10a, is a “heat map” of the expression levels of the overall miR expression. Red indicates that the relative expression of the miR is highly elevated. As this color moves toward pink to white, the expression level is reduced, or suggests no change relative to the control sample. As the color the merges to blue, this indicates that the relative level of the noted miR is significantly reduced. FIG. 10, section A1 of the map indicates that relative to all of the tissues tested that this mir group is highly overexpressed in the stem cells (P<0.001) when compared to any of the tissues tested. Conversely, FIG. 10, section A2 indicates that the miRs comprising this group are highly underexpressed when contrasted with any other tissue (P<0.001). Section 10-A3 indicates that this group of mirs is generally highly over expressed in the other tissues when contrasted with the stem cell miR expression. FIGS. 10B and 10C are the excerpted sections, blown up to visualize the section from FIG. 10A1-3 (Represented by 10-B1, 2 & 10-C3, respectively).

Table 3A, below, indicates the exact miR profiles for the elevated miRs in contrast to the other tissues, while Table 3B, below, indicates the significantly depressed miR signature for the tissue contrasts.

These elevated and repressed expression levels represent the primary although not exclusive starting points for (and combinations thereof) for differentiating the stem cells into clinically valuable alternative phenotypes that may be applied therapeutically.

TABLE 3a
MOST SIGNIFICANTLY ELEVATED MICRORNAs
COMPARED CONTRASTED SPECTRUM OF DIFFERENTIATED
TISSUES CORRECT COUNTERPART

| | ASCENTION | INCREASE (+) |
|---|---|---|
| MIR NUMBER | NUMBER\hat{ } | DECREASE(−) |
| hsa-mir-494-No. 1 | MIMAT0002816 | +++ |
| hsa-mir-346-No. 1 | MIMAT0000773 | +++ |
| hsa_mir_320_Hcd306 left | MI0000542 | +++ |
| hsa-mir-320-No. 2 | MI0000510 | +++ |
| hsa-mir-184-prec-No. 2 | MI0000481 | +++ |
| hsa-mir-328-No. 1 | MIMAT0000752 | +++ |
| hsa-mir-130b-No. 2 | MIMAT0004680 | +++ |
| hsa-mir-196b-No. 2 | MIMAT0001080 | +++ |

\hat{ }Based on Sanger MicroRNA Data Base and Global Terminology and Sequence Standardization http://microrna.sanger.ac.uk

TABLE 3b
MOST SIGNIFICANTLY DEPRESSED MICRORNAs
COMPARED CONTRASTED SPECTRUM OF DIFFERENTIATED
TISSUES CORRECT COUNTERPART

| | ASCENTION | INCREASE (+) |
|---|---|---|
| MIR NUMBER | NUMBER\hat{ } | DECREASE(−) |
| hsa-mir-218-1-prec | MI0000294 | −−− |
| hsa-mir-219-prec | MI0000296 | −−− |
| hsa-mir-425-No. 2 | MIMAT0003393 | −−− |
| hsa-mir-107-No. 1 | MIMAT0000104 | −−− |
| hsa-mir-107-prec-No. 10 | MI0000114 | −−− |
| hsa-mir-103-2-prec | MI0000108 | −−− |
| hsa-mir-103-prec-5=103-1 | MI0000109 | −−− |
| hsa-mir-15a-No. 1 | MIMAT0000068 | −−− |
| hsa-mir-15^{a}-2-prec-No. 1 | MI0000069 | −−− |
| hsa-mir-15b-prec-No. 1 | MI0000438 | −−− |
| hsa-mir-451-No. 1 | MIMAT0001631 | −−− |
| hsa-mir-123-prec-No. 2 | MI000471 | −−− |
| hsa-mir-126*-No. 2 | MIMAT0000444 | −−− |
| hsa-mir-126-No. 1 | MIMAT0000445 | −−− |
| hsa-mir-195-prec | MI0000489 | −−− |
| hsa-mir-16-2-No. 1 | MIMAT0000069/115 | −−− |
| hsa-mir-16-1-No. 1 | MIMAT0000069/70 | −−− |
| hsa-mir-17-prec-No. 1 | MI0000071 | −−− |
| hsa-mir-16^{a}-chr13 | MIMAT0000070/x4802 | −−− |
| hsa-mir-16b-chr3 | MIMAT0000115/x4919 | −−− |
| hsa-mir-19^{a}-prec | MI0000073 | −−− |
| hsa-mir-194-prec-No. 2 | MI0000732 | −−− |
| hsa-mir-342-No. 2 | MIMAT0004694 | −−− |
| hsa-mir-429-No. 2 | MIMAT0001536 | −−− |
| hsa-mir-218-2-prec-No. 2 | MI0000295 | −−− |
| hsa-let 7f-1-prec-No. 1 | MI0000067 | −−− |
| hsa-mir-188-prec | MI0000484 | −−− |
| hsa-mir-203-prec-No. 1 | MI0000283 | −−− |
| hsa-mir-205-prec | MI0000285 | −−− |
| hsa-mir-023a-prec | MI0000079 | −−− |
| hsa-mir-023b-prec | MI0000439 | −−− |
| hsa-mir-024-1-prec-No. 1 | MI0000080 | −−− |
| hsa-mir-024-2-prec | MI0000081 | −−− |
| hsa-mir-027a-prec | MI0000085 | −−− |
| hsa-mir-027b-prec | MI0000440 | −−− |
| hsa-mir-021-prec17-No. 1 | MI0000077 | −−− |
| hsa-mir-021-No. 1 | MIMAT0000076 | −−− |
| hsa-mir-187-prec-No. 1 | MI0000274 | −−− |
| hsa-mir-143-prec | MI0000459 | −−− |
| hsa-mir-145-prec | MI0000461 | −−− |
| hsa-mir-031-prec | MI0000089 | −−− |
| hsa-mir-194-2-No. 1 | MI0000488 | −−− |
| hsa-mir-215-prec-No. 2 | MI0000291 | −−− |
| hsa-mir-192-2/3-No. 1 | MIMAT0000222 | −−− |
| hsa-mir-192-No. 1 | MIMAT0000222 | −−− |
| hsa-mir-215-prec-No. 1 | MI0000291 | −−− |
| hsa-mir-375 | MIMAT0000728 | −−− |
| hsa-mir-141-prec-No. 1 | MI0000457 | −−− |
| hsa-mir-200a-prec | MI0000737 | −−− |
| hsa-mir-200b-No. 1 | MIMAT000318 | −−− |
| hsa-mir-192-/3-No. 2 | MIMAT0000222 | −−− |
| hsa-mir-370-No. 2 | MIMAT0000722 | −−− |
| hsa-mir-152-prec-No. 1 | MI0000462 | −−− |
| hsa-mir-148b-No. 1 | MIMAT0000759 | −−− |
| hsa-mir-148a-No. 1 | MIMAT0000722 | −−− |
| hsa-mir-148-prec | MIMAT0000243 | −−− |
| hsa-mir-206-prec-No. 1 | MI0000490 | −−− |
| hsa-mir-001b-1-prec-No. 1 | MI0000651 | −−− |
| hsa-mir-133a-1 | MIMAT0000427 | −−− |
| hsa-mir-007-1-prec | MI0000263 | −−− |

\hat{ }Based on Sanger MicroRNA Data Base and Global Terminology and Sequence Standardization http://microrna.sanger.ac.uk

### INDUSTRIAL APPLICABILITY

The applicability of the invention is in the area of medical arts, specifically, the diagnosis, evaluation and treatment of the mammalian conditions, including human beings (Homo sapien sapien), through the use of stem cells and microRNAs as uniquely elucidated and described herein.

What is claimed is:

1. A stem cell microRNA based method for diagnosis, evaluation and treatment of mammalian conditions, pathology, and diseases comprising the steps of:
(a) measuring the level of at least one miR gene product in a biological sample derived from a subject's tissue or tissue product;
(b) comparing the level of said at least one miR gene product in the biological sample to the level of a corresponding at least one miR gene product in a control sample, said control sample comprising an adult stem cell, wherein a detected difference between the level of said at least one miR gene product in the biological sample as compared with the level of said corresponding at least one miR gene product in the control sample is indicative of the presence or absence of adult stem cells; and
(c) therapeutically applying at least one miR gene product in vivo, in vitro or a combination of in vitro and in vivo methods.

2. The method of claim 1, wherein said therapeutically applying step includes therapeutically applying more than one miR gene product.

3. The method of claim 1, wherein said at least one miR gene product measured in step (a) is selected from the group consisting of:
hsa-mir-517b
hsa-mir-517c
hsa-mir-517a
hsa-mir-519a-1
hsa-mir-519a-2Nol
hsa-mir-520gNol
hsa-mir-135bNo1
hsa_mir_{—}95 right
hsa-mir-205-prec
hsa-mir-383No1
hsa-mir-022-prec
hsa-mir-509No1
hsa-mir-188-prec
hsa-mir-187-precNol
hsa-mir-200cNol
hsa-mir-222-precNol
hsa-mir-211-precNo2
hsa-mir-519dNol
hsa-mir-095-prec-4
hsa-mir-338No1
hsa-mir-009-3No2
hsa-mir-127-prec
hsa-mir-001b-1-precl
hsa-mir-26a-2Nol
hsa-mir-203-precNol
hsa-mir-199b-precNo2
hsa-mir-029c-prec
hsa-mir-105-2No2
hsa-mir-326No2
hsa-mir-519b
hsa-mir-106-prec-X
hsa-mir-425No1
hsa-mir-105-2Nol
hsa-mir-369No1
hsa-mir-132-precNo2
hsa-mir-181b-2No2
hsa-mir-030b-precNol
hsa-mir-100Nol
hsa-mir-026b-prec
hsa-mir-015b-precNol
hsa-mir-345No2
hsa-mir-030a-precNo2
hsa-mir-020-prec
hsa-mir-328No1
hsa-mir-192-2/3Nol
hsa-mir-149-prec
hsa-mir-30eNo2
hsa-mir-200bNol
hsa-miR-126*Nol
hsa-mir-486
hsa-mir-212-precNol
hsa-mir-499No1
hsa-mir-337No1
hsa-mir-321No1
hsa-mir-139-prec
hsa-mir-125b-2-precNo2
hsa-mir-100-1/2-prec
hsa-mir-208-prec
hsa-mir-030c-prec
hsa-mir-126No2
hsa-mir-410Nol
hsa-mir-450-1
hsa-mir-196-1-precNo2
hsa-mir-030a-precNol
hsa-mir-I 84-precNo2
hsa-mir-030d-precNo2
hsa-mir-133 bNo2
hsa-mir-30c-1Nol
hsa-mir-361Nol
hsa-mir-30c-2Nol
hsa-mir-29b-1Nol
hsa-mir-321Not
hsa-mir-192No1
hsa-mir-491No2
hsa-miR-373*Nol
hsa_mir_{—}320_Hcd306 right
hsa-mir-335No 1
hsa-mir-I 81c-precNol
hsa-mir-500No2
hsa-mir-346No1
hsa-mir-485-3p
hsa-mir-155-prec
hsa-mir-30eNol
hsa-mir-218-2-precNo2
hsa-mir-099-prec-21
hsa-mir-102-prec-1
hsa-mir-101-1/2-precNol
hsa-mir-130bNol
hsa-mir-422allo2
hsa-mir-330No2
hsa-mir-010a-precNol
hsa-mir-017-precNo2
hsa-mir494No1
hsa-mir-26a-1Nol
hsa-mir-130bNo2
hsa-mir-034-precNo2
hsa-mir-345No1
hsa-mir-219-1Nol
hsa-mir-374No1
hsa-mir-148allol
hsa-mir-373No2
hsa-mir-106allol
hsa-mir-423No2
hsa-mir-199a-1-prec
hsa-mir-30c-1No2
hsa-mir-505No1
hsa-mir-324No2
hsa-mir-224-prec
hsa-mir-335No2
hsa-mir-330No1
hsa-mir-206-precNol
hsa-mir-027a-prec
hsa-mir-026a-precNol
hsa-mir-193-precNo2
hsa-mir-007-2-precNol
hsa-mir-222-precNo2
hsa-mir-199b-precNol
hsa-mir-106bNol
hsa-mir-200a-prec
hsa-mir-123-precNol
hsa-mir-29b-2=102prec7.1=7.2
hsa-mir-129-2No2
hsa-mir-181b-1No2
hsa-mir-326No1
hsa-mir-185-precNo2
hsa-mir-147-prec
hsa-mir-134-precNo2
hsa-mir-140No2
hsa-mir-361No2
hsa-mir-148bNol
hsa-mir-493No2
hsa-mir-299No1
hsa-mir-365-2No2
hsa-mir-181c-precNo2
hsa-mir-215-precNol
hsa-mir-135-1-prec
hsa-mir-521-1Nol
hsa-mir-125a-precNo2
hsa-mir-202-prec
hsa-mir-16-2No2
hsa-mir-375
hsa-miR-324-5pNo2
hsa-mir-029a-2Nol
hsa-mir-144-precNol
hsa-mir-030d-precNol
hsa-mir-429No2
hsa-mir-030b-precNo2
hsa-mir-130a-precNo2
hsa-mir-007-3-precNo1
hsa-mir-424No2
hsa-mir-211-precNol
hsa-mir-500Nol
hsa-mir-320Nol
hsa-mir-009-3Nol
hsa-mir-033-prec
hsa-mir-019b-2-prec
hsa-mir-194-2No1
hsa-mir-20bNo1
hsa-mir-034precNol
hsa-mir-146bNo1
hsa-mir-195-prec
hsa-mir-032-precNol
hsa-mir-129-precNo2
hsa-mir-206-precNo2
hsa-mir-130a-precNo 1
hsa-mir-331No2
hsa-mir-34allo1
hsa-mir-181a-precNol
hsa-mir-032-precNo2
hsa-mir-34bNo2
hsa-mir-324No1
hsa-mir-521-2Nol
hsa-mir-489Nol
hsa-mir-30c-2No2
hsa-mir-516-1No 1
hsa-mir-504No2
hsa-mir-009-1Nol
hsa-mir-342No1
hsa-mir-135a-2No2
hsa-mir-380-5p
hsa-mir-450-2Nol
hsa-mir-518bNol
hsa-mir-197-prec
hsa-mir-519c
hsa-let-7c-prec
hsa-mir-299No2
hsa-mir-101-2No2
hsa-mir-516-2Nol
hsa-mir-448No1
hsa-let-71No2
hsa-let-7f-1-precNol
hsa-let-7a-l-prec
hsa-mir-525
hsa-mir-485-5p
hsa-mir-519a-2No2
hsa-mir-520gNo2
hsa-mir-382
hsa-mir-502Nol
hsa-mir-216-precNo2
hsa-mir-196bNo2
hsa-mir-424No1
hsa-mir-203-precNo2
hsa-mir-021-prec-17No1
hsa-mir-520a*
hsa-let-7b-prec
hsa-mir-515-13p
hsa-mir-378No2
hsa-mir-502No2
hsa-mir-4323p
hsa-mir-183-precNo2
hsa-mir-153-1-prec2
hsa-mir-52OhNo1
hsa-mir-128b-precNol
hsa-mir-498No2
hsa-mir-329-2No2
hsa-mir429No1
hsa-mir-199a-2-prec
hsa-let-7iNol
hsa-mir-513-2No2
hsa-mir-513-2Nol
hsa-mir-302bNo2
hsa-mir-129-precNol
hsa-mir-507No1
hsa-mir-433No2
hsa-mir-511-1No2
hsa-mir-152-precNo2
hsa-mir-202
hsa-mir-296No 1
hsa-mir-186-prec
hsa-mir-52OhNo2
hsa-mir-210-prec
hsa-let-7f-1-precNo2
hsa-mir-099b-prec-19Nol
hsa-mir-153-1-precl
hsa-mir-450-2No2
hsa-mir-362No1
hsa-mir-511-1Nol
hsa-mir-513-1Nol
hsa-mir-367No1
hsa-mir-193bNo2
hsa-mir-192No2
hsa-mir-515-23p
hsa-mir-34bNol
hsa-mir-150-prec
hsa-mir-128a-precNol
hsa-mir-027b-prec
hsa-mir-495No2
hsa_mir_{—}95 left
hsa-mir-181b-2Nol
hsa-mir-449Nol
hsa-mir-028-prec
hsa-mir-105-prec-X.1=105-1
hsa-mir-216-precNol
hsa-mir-137-prec
hsa-mir-524
hsa-let-7d-prec
hsa-mir-019b-1-prec
hsa-mir-138-2-prec
hsa-mir-219-prec
hsa-mir-412No1
hsa-mir-16-2Nol
hsa-mir-487No1
hsa-mir-145-prec
hsa-mir-21Nol
hsa-mir-363No2
hsa-mir-518a-2*
hsa-mir-101-1/2-precNo2
hsa-mir-181b-precNol
hsa-mir-141-precNo2
hsa-mir-26a-2No2
hsa-mir-018-prec
hsa-mir-381Nol
hsa-mir-092-prec-13492-1No2
hsa-mir-025-prec
hsa-mir-453No1
hsa-mir-342No2
hsa-let-7a-3-prec
hsa-mir-503Nol
hsa-mir-302allol
hsa-mir-518c*
hsa-mir-092-prec-134192-1Nol
hsa-mir-377No2
hsa-mir-154-precl Not
hsa-mir-218-2-precNol
hsa-let-7e-prec
hsa-mir-132-precNol
hsa-mir-483No2
hsa-mir-520fNo1
hsa-mir-016a-chr13
hsa-mir-525*
hsa-mir-146-prec
hsa-mir-135bNo2
hsa-mir-007-2-precNo2
hsa-mir-431No2
hsa-mir-202*
hsa-mir-363No1
hsa-mir-496No2
hsa-mir-16-1Nol
hsa-mir-204-precNol
hsa-mir-433No1
hsa-mir-122a-prec
hsa-mir-101-prec-9
hsa-mir-339No2
hsa-mir-495No1
hsa-mir-409-5p
hsa-mir-010b-precNo2
hsa-mir-519dNo2
hsa-mir-325No2
hsa-mir-181b-1 Nol
hsa-mir-193bNol
hsa-mir-125a-precNol
hsa_mir_{—}147 left
hsa-mir-338No2
hsa-mir-016b-chr3
hsa-mir-221-prec
hsa-mir-213-precNol
or combinations thereof.

4. The method of claim 1, wherein the level of the miR gene product in the biological sample is greater than the level of the corresponding miR gene product in the control sample.

5. The method of claim 4, wherein the at least one miR gene product is selected from the group consisting of:
hsa-mir-030b-precNol
hsa-mir-338No1
hsa-mir-329-2Nol
hsa-mir-106-prec-x
hsa-mir-26a-1Nol
hsa-mir-337No1
hsa-mir-335No2
hsa-mir-192No2
hsa-mir-135bNo1
hsa_mir_{—}95 right
hsa-mir-211-precNo2
hsa-mir-095-prec-4
hsa-mir-338No1
hsa-mir-26a-2Nol
hsa-mir-199b-precNo2
hsa-mir-029c-prec
hsa-mir-106-prec-X
hsa-mir-425No1
hsa-mir-105-2No1
hsa-mir-030b-precNol
hsa-mir-100Nol
hsa-mir-026b-prec
hsa-mir-030a-precNo2
hsa-mir-020-prec
hsa-mir-328No1
hsa-mir-149-prec
hsa-miR-126*Nol
hsa-mir-212-precNol
hsa-mir-100-1/2-prec
hsa-mir-030c-prec
hsa-mir-030a-precNol
hsa-mir-30c-1Nol
hsa-mir-30c-2Nol
hsa-mir-491No2
hsa-mir-099-prec-21
or combinations thereof.

6. The method of claim 1, wherein the level of said at least one miR gene product measured in step (a) is less than the level of said corresponding said at least one miR gene product in the control sample.

7. The method of claim 6, wherein said at least one miR gene product measured in step (a) is selected from the group consisting of:
hsa-mir-521-1Nol
hsa-mir-516-2Nol
hsa-mir-52OhNol
hsa-mir-519a-2No2
hsa-mir-521-2Nol
hsa-mir-202-prec
hsa-mir-155-prec
hsa-mir-144-precNol
hsa-mir-375
hsa-mir-515-13p
hsa-mir-517b
hsa-mir-517c
hsa-mir-517a
hsa-mir-519a-1
hsa-mir-519a-2No1
hsa-mir-520gNol
hsa-mir-205-prec
hsa-mir-383No1
hsa-mir-022-prec
hsa-mir-509No1
hsa-mir-188-prec
hsa-mir-187-precNol
hsa-mir-200cNol
hsa-mir-222-precNol
hsa-mir-519dNo1
hsa-mir-009-3No2
hsa-mir-127-prec
hsa-mir-001b-1-precl
hsa-mir-203-precNol
hsa-mir-326No2
hsa-mir-519b
hsa-mir-486
hsa-mir-321Nol
hsa-mir-196-1-precNo2
hsa-mir-321No2
or combinations thereof.

8. The method of claim 1, wherein said at least one miR gene product measured in step (a) is selected from the group consisting of:
hsa-mir-001b-1-prec1
hsa-let-7a-2-precNo1
hsa-let-7g-precNo2
hsa-let-7f-1-precNo1
hsa-mir-009-3No2
hsa-mir-010a-precNo1
hsa-mir-015b-precNo1
hsa-mir-019b-1-prec
hsa-mir-020-prec
hsa-mir-021-prec-17No1
hsa-mir-022-prec
hsa-mir-26a-1No1
hsa-mir-26a-2No1
hsa-mir-026b-prec
hsa-mir-027a-prec
hsa-mir-29b-
hsa-mir-029c-prec
hsa-mir-030a-precNo2
hsa-mir-030b-precNo1
hsa-mir-030c-prec
hsa-mir-30c-1No1
hsa-mir-30c-2No1
hsa-mir-095-prec-4
hsa-mir-099b-prec-19No1
hsa-mir-099-prec-21
hsa-mir-100-1/2-prec
hsa-mir-100No1
hsa-mir-102-prec-1
hsa-mir-105-2No1
hsa-mir-106-prec-X
hsa-mir-123-precNo1
hsa-mir-125b-2-precNo2
hsa-mir-126No1
hsa-mir-127-prec
hsa-mir-130a-precNo2
hsa-mir-130bNo2
hsa-mir-133bNo2
hsa-mir-135bNo1
hsa-mir-140No2
hsa-mir-141-precNo1
hsa-mir-144-precNo1
hsa-mir-149-prec
hsa-mir-155-prec
hsa-mir-181a-precNo1
hsa-mir-181c-precNo1
hsa-mir-187-precNo1
hsa-mir-188-prec
hsa-mir-192-2/3No1
hsa-mir-192No1
hsa-mir-192No2
hsa-mir-199b-precNo1
hsa-mir-199b-precNo2
hsa-mir-200bNo1
hsa-mir-202-prec
hsa-mir-203-precNo1
hsa-mir-205-prec
hsa-mir-211-precNo2
hsa-mir-222-precNo1
hsa-mir-299No1
hsa-mir-302bNo1
hsa-mir-324No2
hsa-mir-326No2
hsa-mir-328No1
hsa-mir-329-2No1
hsa-mir-335No1
hsa-mir-335No2
hsa-mir-337No1
hsa-mir-338No1
hsa-mir-345No1
hsa-mir-345No2
hsa-mir-361No1
hsa-mir-369No1
hsa-miR-373*No1
hsa-mir-375
hsa-mir-383No1
hsa-mir-409-3p
hsa-mir-425No1
hsa-mir-450-1
hsa-mir-499No1
hsa-mir-509No1
hsa-mir-515-13p
hsa-mir-516-2No1
hsa-mir-517a
hsa-mir-517b
hsa-mir-517c
hsa-mir-519a-1
hsa-mir-519a-1/526c
hsa-mir-519a-2No2
hsa-mir-519b
hsa-mir-519dNo1
hsa-mir-520gNo1
hsa-mir-520hNo1
hsa-mir-521-1No1
hsa-mir-521-2No1
or combinations thereof.

9. The method of claim 1, wherein said at least one miR gene product measured in step (a) is selected from the group consisting of:
hsa-mir-009-3No2
hsa-mir-015b-precNo1
hsa-mir-019b-1-prec
hsa-mir-020-prec
hsa-mir-021-prec-17No1
hsa-mir-022-prec
hsa-mir-26a-1No1
hsa-mir-26a-2No1
hsa-mir-026b-prec
hsa-mir-027a-prec
hsa-mir-029c-prec
hsa-mir-30c-1No1
hsa-mir-30c-2No1
hsa-mir-099b-prec-19No1
hsa-mir-099-prec-21
hsa-mir-100No1
hsa-mir-102-prec-1
hsa-mir-105-2No1
hsa-mir-106-prec-X
hsa-mir-125b-2-precNo2
hsa-mir-126No1
hsa-mir-127-prec
hsa-mir-130a-precNo2
hsa-mir-130bNo2
hsa-mir-135bNo1
hsa-mir-140No2
hsa-mir-181a-precNo1
hsa-mir-181c-precNo1
hsa-mir-187-precNo1
hsa-mir-188-prec
hsa-mir-192No2
hsa-mir-199b-precNo2
hsa-mir-205-prec
hsa-mir-222-precNo1
hsa-mir-299No1
hsa-mir-302bNo1
hsa-mir-324No2
hsa-mir-326No2
hsa-mir-328No1
hsa-mir-335No1
hsa-mir-337No1
hsa-mir-361No1
hsa-mir-383No1
hsa-mir-425No1
hsa-mir-450-1
hsa-mir-509No1
hsa-mir-517a
hsa-mir-517b
hsa-mir-517c
hsa-mir-519a-1
hsa-mir-519a-1/526c
hsa-mir-519a-2No2
hsa-mir-519b
hsa-mir-519dNo1
hsa-mir-520g No1
hsa-mir-520hNo1
hsa-mir-521-1No1
hsa-mir-521-2No1
hsa-mir-009-3No2
hsa-mir-015b-precNo1
hsa-mir-019b-1-prec
hsa-mir-020-prec
hsa-mir-021-prec-17No1
hsa-mir-022-prec
hsa-mir-26a-1No1
hsa-mir-26a-2No1
hsa-mir-026b-prec
hsa-mir-027a-prec
hsa-mir-029c-prec
hsa-mir-30c-1No1
hsa-mir-30c-2No1
hsa-mir-099b-prec-19No1
hsa-mir-099-prec-21
hsa-mir-100No1
hsa-mir-102-prec-1
hsa-mir-105-2No1
hsa-mir-106-prec-X
hsa-mir-125b-2-precNo2
hsa-mir-126No1
hsa-mir-127-prec
hsa-mir-130a-precNo2
hsa-mir-130bNo2
hsa-mir-135bNo1
hsa-mir-140No2
hsa-mir-181a-precNo1
hsa-mir-181c-precNo1
hsa-mir-187-precNo1
hsa-mir-188-prec
hsa-mir-192No2
hsa-mir-199b-precNo2
hsa-mir-205-prec
hsa-mir-222-precNo1
hsa-mir-299No1
hsa-mir-302bNo1
or combinations thereof.

10. The method of claim 1, wherein said at least one miR gene product measured in step (a) is selected from the group consisting of:
hsa-mir-015b-precNo1
hsa-mir-019b-1-prec
hsa-mir-026b-prec
hsa-mir-027a-prec
hsa-mir-30c-1No1
hsa-mir-30c-2No1
hsa-mir-099-prec-21
hsa-mir-100No1
hsa-mir-105-2No1
hsa-mir-106-prec-X
hsa-mir-125b-2-precNo2
hsa-mir-126No1
hsa-mir-130a-precNo2
hsa-mir-135bNo1
hsa-mir-187-precNo1
hsa-mir-188-prec
hsa-mir-192No2
hsa-mir-205-prec
hsa-mir-222-precNo1
hsa-mir-302bNo1
hsa-mir-335No1
hsa-mir-337No1
hsa-mir-450-1
hsa-mir-509No1
hsa-mir-517a
hsa-mir-517b
hsa-mir-517c
hsa-mir-519a-1
hsa-mir-519a-1/526c
hsa-mir-519a-2No2
hsa-mir-519b
hsa-mir-519dNo1
hsa-mir-520gNo1
hsa-mir-520hNo1
hsa-mir-521-1No1
hsa-mir-521-2No1
or combinations thereof.
